(19) United States
(12) Patent Application Publication
(10) Pub. No.: US 2024/0341588 A1
(43) Pub. Date: Oct. 17, 2024

(54) OPTICAL COHERENCE TOMOGRAPHY SYSTEM AND METHOD FOR IMAGING OF A SAMPLE

(71) Applicant: NANYANG TECHNOLOGICAL UNIVERSITY, Singapore (SG)

(72) Inventor: Linbo LIU, Singapore (SG)

(73) Assignee: NANYANG TECHNOLOGICAL UNIVERSITY, Singapore (SG)

(21) Appl. No.: 18/579,998

(22) PCT Filed: Aug. 10, 2022
(86) PCT No.: PCT/SG2022/050570

§ 371 Date: Jan. 17, 2024

(30) Foreign Application Priority Data
Aug. 10, 2021 (SG) 10202108701U
Dec. 23, 2021 (SG) 10202114280W

(51) Int. Cl. A61B 3/10; A61B 3/12; G01B 9/02091

(52) U.S. Cl. CPC A61B 3/102; A61B 3/1241; G01B 9/02091

(57) ABSTRACT

Aspects concern an optical coherence tomography (OCT) system for imaging of a sample, comprising: a sample arm for directing light onto the sample, the sample arm comprises sample arm optics comprising a dispersive element to generate an extended source for illuminating the sample: a reference arm: a detector for detecting an interference signal from light that is reflected from the reference arm and light that is back-reflected or back-scattered from the sample; and a scanner for scanning the extended source across the sample along a fast axis and a slow axis such that a plurality of partial-spectrum frames is obtained at the detector: wherein the dispersive element is orientable such that the extended source is disposed at a non-zero angle to the fast axis.

### CROSS-REFERENCE TO RELATED APPLICATION

The application claims the benefit of priority of Singapore patent application Ser. No. 10202108701U filed on 10 Aug. 2021 & 10202114280 W filed on 23 Dec. 2021, the content of it being hereby incorporated by reference in its entirety for all purposes.

### TECHNICAL FIELD

The disclosure relates to optical coherence tomography system and method for imaging a sample.

### BACKGROUND

Optical coherence tomography (OCT) is an established in vivo optical imaging technology that provides micrometer resolution and millimeter penetration depth in human tissues. Since its invention in 1991 October technology has evolved from a time-domain OCT (TD-OCT) as a first-generation technology to a Fourier-domain (FD-OCT) as a second-generation technology. FD-OCT technology includes spectral domain OCT (SD-OCT) and swept source OCT (SS-OCT) or optical frequency domain imaging (OFDI).

Over the years, many OCT-based techniques have been developed for various purposes. In particular, OCT angiography (OCTA) have been developed to image vascular structure and is adapted to allow visualization of blood vessels in a living tissue. Part of the OCTA process requires repeated sampling of the same x-z plane in a time-lapsed manner (B-M mode), also referred to as repeated B-scans, to account for blood flow in the blood vessels. There are other OCT-based techniques that require repeated B-scans such as phase-sensitive OCT, OCE, dynamic OCT. However, existing OCT-based techniques inadequately take into account varying speed of blood flow within blood vessels, and motion artifact between repeated scans, for example, pulsatile expansion and contraction of arteries, saccades of imaging samples such as eyes.

Although various methods have been proposed to minimize motion artifact, via, for example, postprocessing corrections, such postprocessing corrections may in turn introduce new artifacts and increase image acquisition time and difficulty. As an alternative approach to artifact minimization, real-time tracking system has been proposed but such solutions require additional imaging hardware which can increase costs and complexity of the OCT system.

There exists a need to provide an improved solution to mitigate at least one of the above limitations or drawbacks.

### SUMMARY

A technical solution in the form of an OCT system with adjustable or orientable dispersive element such that the extended source is disposed at a non-zero angle with respect to a scanning axis, is proposed. Such an arrangement provides a relatively non-complex solution to obtain a robust set of partial-spectrum frames, for example, amplitude, phase or complex (APC) frames for processing. In addition, the technical solution provides for various methods of processing to improve field-of-view, imaging speed, accuracy, minimize and/or correct motion artifacts, and achieve dynamic inter-scan time.

According to an aspect of the present disclosure, an optical coherence tomography (OCT) system for imaging of a sample is provided. The OCT system comprises a sample arm for directing light onto the sample, the sample arm comprises sample arm optics comprising a dispersive element to generate an extended source for illuminating the sample; a reference arm; a detector for detecting an interference signal from light that is reflected from the reference arm and light that is back-reflected or back-scattered from the sample; and a scanner for scanning the extended source across the sample along a fast axis and a slow axis such that a plurality of partial-spectrum frames is obtained at the detector; wherein the dispersive element is orientable such that the extended source is disposed at a non-zero angle to the fast axis.

In an embodiment, the non-zero angle is an acute angle.

In an embodiment, the non-zero angle is a right angle.

In an embodiment, the scanner is configured to scan a plurality of times along the fast axis at each of a respective plurality of positions along the slow axis.

In an embodiment, the system further comprises at least one processor configured to generate at least one OCT and/or at least one OCTA image from the partial-spectrum frames. The at least one processor may be configured to check for at least one low quality frame of the partial-spectrum frames and remove the at least one low quality frame prior to generating the at least one OCT image and/or the at least one OCTA image.

In an embodiment, the at least one processor is configured to perform temporal averaging of the partial-spectrum frames for each of the respective plurality of positions to generate a plurality of respective OCT images associated with the respective plurality of positions.

In an embodiment, the processor is configured to perform frequency compounding of the partial-spectrum frames for each of the respective plurality of positions.

In an embodiment, each of the respective plurality of positions along the slow axis is set at an inter-scan distance L of 1≤L≤P, wherein P is the number of partial-spectrum frames.

In an embodiment, the system comprises at least one processor configured to obtain or derive depth-axis scan information corresponding to the scanning along the fast axis and/or the slow axis, and wherein an inter-scan time between each of the plurality of times is adjustable by varying the period of the depth-axis scan with respect to the slow axis, and/or by varying a number of times the scanner moves along the fast axis and/or the slow axis.

In an embodiment, the sample is an eye.

According to another aspect of the present disclosure, there is a method for optical coherence tomography for imaging of a sample, comprising the steps of: disposing a dispersive element in a sample arm of an optical coherence tomography system to generate an extended source for illuminating the sample; scanning the extended source across the sample along a fast axis and a slow axis, whereby a plurality of partial-spectrum frames is obtained; and detecting an interference signal generated by light received from the sample arm and light received from a reference arm of the optical coherence tomography system; wherein the dispersive element is oriented such that the extended source is disposed at a non-zero angle to the fast axis.

In an embodiment, the non-zero angle is an acute angle.

In an embodiment, the non-zero angle is a right angle.

In an embodiment, the step of scanning comprises scanning a plurality of times along the fast axis at each of a respective plurality of positions along the slow axis.

In an embodiment, the method further comprises a step of generating at least one OCT image and/or at least one OCTA from the partial-spectrum frames.

In an embodiment, the method further comprises a step of removing low quality frames of the partial spectrum frames prior to generating the at least one OCT image and/or the at least one OCTA image.

In an embodiment, the step of generating the OCT image comprises performing temporal averaging of the partial-spectrum frames for each of the respective plurality of positions to generate a plurality of respective OCT images for the respective plurality of positions.

In an embodiment, the method further comprises a step of performing frequency compounding of the partial-spectrum frames for each of the respective plurality of positions.

In an embodiment, each of the respective plurality of positions along the slow axis is set at an inter-scan distance L of 1≤L≤P, wherein P is the number of partial-spectrum frames.

In an embodiment, the step of scanning further comprises scanning, obtaining or deriving a depth-axis in combination with the fast axis and/or the slow axis, and wherein an inter-scan time between each of the plurality of times is adjustable by varying the period of scanning the depth-axis with respect to the slow axis and/or by varying a number of times the scanner moves along the fast axis and/or the slow axis.

In an embodiment, the sample is an eye.

According to another aspect of the present disclosure there is a method for modifying an optical coherence tomography (OCT) system for imaging of a sample, the OCT system comprising a sample arm for directing light onto the sample, a reference arm, a detector for detecting an interference signal from light that is reflected from the reference arm and light that is back-reflected or back-scattered from the sample, comprising the steps of: disposing a dispersive element in the sample arm to generate an extended source for illuminating the sample; configuring the scanner to scan the extended source across the sample along a fast axis and a slow axis such that a plurality of partial-spectrum frames is obtained at the detector; wherein the dispersive element is orientable such that the extended source is disposed at a non-zero angle to the fast axis.

### BRIEF DESCRIPTION OF THE DRAWINGS

The disclosure will be better understood with reference to the detailed description when considered in conjunction with the non-limiting examples and the accompanying drawings, in which:

FIG. 1 shows a schematic diagram or view of a prior art optical coherence tomography (OCT) system.

FIG. 2A shows a schematic view of an OCT system according to various embodiments of the present disclosure.

FIG. 2B shows a schematic three-dimensional view of a sample arm of the OCT of FIG. 2A.

FIG. 2C shows a schematic view of another OCT system according to various embodiments of the present disclosure.

FIG. 2D shows a schematic view of an endoscopic probe in accordance with another embodiment of the present disclosure.

FIG. 3A and FIG. 3B depicts scanning pattern of point source OCT and an exemplary scanning pattern of the OCT system shown in FIG. 2A respectively.

FIG. 4A illustrates a process to generate partial-spectrum APC variation frames according to various embodiments.

FIG. 4B illustrates a frequency compounding schematic performed by a processor of the system shown in FIG. 2A.

FIG. 4C is a plot of a measured axial resolution.

FIG. 4D shows a plot of a 10-90% edge width of the monochromatic beam, spectral-window convoluted beam, and deconvolution result.

FIG. 4E shows an US Air Force 1951 Resolution chart images before (top) and after (bottom) deconvolution.

FIG. 5 shows the sample arm of the OCT system of FIG. 2A where the dispersive element is orientable such that the extended source is disposed at an acute angle to the fast axis (B1, BC, and BL represent the principle ray of the collimated beam of λ₁, λ_{c} and λ_{L}, respectively, where λ₁, λ_{c} and λ_{L} are short cut-off wavelength, center wavelength, and long cut-off wavelength of the broadband source spectrum, respectively).

FIG. 6 illustrates the division of an illumination spectrum into P equally spaced spectral bands, and P number of partial-spectrum APC variation frames obtained from spectral interference data acquired in one B-scan.

FIG. 7 illustrates the division of scan areas of an extended source in the X-Y plane of multiple repeated-scans-at-the-same-y-position (RBSSYP).

FIG. 8A is a flow chart depicting a process 800 for processing the scanned image data collected.

FIG. 8B shows an exemplary spectral signal remapping algorithm according to step 802 of the process 800.

FIG. 9A shows an OCT angiogram of human inner retina (OD) using the point-scanning scheme (A), FIG. 9B shows the corresponding OCT structural image, FIG. 9C to FIG. 9E show an OCT angiogram acquired with a scanning scheme of the present disclosure covering both the macula and the temporal side of optic disc before (FIG. 9C) & after (FIG. 9D) motion correction, and the corresponding OCT structural image (FIG. 9E). (A straight white line is added at the top of the uncorrected imaging, which reflects the vertical motion trajectory in the corrected image (arrowheads, FIGS. 9C & 9D). Arrows indicate correction of vessel disruption. Asterisk indicates an uncorrected vessel disruption. Scale bars: 500 μm.

FIG. 10A shows a photography of a sample in the form of a human skin with a thin, straight metal wire (arrow) attached, and an OCT intensity cross-sectional image showing the shadow of the metal wire. With dotted box defining an area for OCT scanning by the system of the present disclosure, FIG. 10B shows fourteen en face angiograms with each generated from one RBSSYP, with arrow indicating an air bubble, FIGS. 10C and 10D show motion data measured along X and Y directions, respectively, FIG. 10E shows an absolution local motion calculated as square root of sum of relative local motions squared, FIGS. 10F and 10G show an en face angiogram before motion correction, and after motion correction, respectively. Stars in the various drawings indicate the location of the metal wire shadow, arrowheads indicate vessel bifurcations, and arrows indicate an air bubble. Scale bar: 1 mm.

FIG. 11A illustrates a Fast (X) and slow (Y) axis scanning position for realizing two different inter-scan time by use of partial B-scans according to some embodiments, FIG. 11B illustrates an OCTA en face image generated by averaging 4 frames with inter-scan time of 5 ms, FIG. 11C illustrates an OCTA en face image generated by averaging 4 frames with inter-scan time of 10 ms, FIG. 11D illustrates an OCTA en face image combining data of both inter-scan time demonstrating larger flow dynamic range. Arrows indicate vessels with higher flow, which is not reflected in FIG. 11C.

FIG. 11E shows another embodiment or method for Fast (X) and slow (Y) axis scanning position for realizing 2 different inter-scan time by use of 2 different A-line period.

FIG. 11F shows a simplified model to reconstruct angiograms with high dynamic range based on the inter-scan time of 11E.

FIG. 11G is a histogram showing image gray level acquired from FIGS. 11B to 11D.

FIG. 12 is a plot of an amplitude decorrelation of inter-scan time T and T/2, and the dynamic range improvement.

FIG. 13 shows a human retina image captured in vivo by the OCT system of the present disclosure. The labels A1, B1, C1 and D1 relate to en face OCTA images of four inter-scan time: 0.54 ms. 1.35 ms, 2.7 ms, and 5.4 ms, respectively.

FIG. 14 shows a high dynamic range OCTA image of a human retina in vivo merged from images of four inter-scan time of FIG. 13.

FIGS. 15A and 15B show an output image of a sample in the form of an en face blood vessel network of human skin in vivo generated by the OCT system of the present disclosure without time averaging and with time average (K=8, L=2) respectively (with image size: 5.6 mm by 3.25 mm).

FIGS. 16A and 16B show an en face blood vessel network of human skin in vivo generated by the OCT system of the present disclosure with time averaging (K=8, L=2), where FIG. 16A is an OCTA image with residual motion artifacts (in arrow heads), and FIG. 16B is the OCTA image after rejecting relatively low-quality partial-spectrum OCTA frames (with Image size: 5.6 mm by 3.25 mm).

FIG. 17 shows an en face blood vessel network of human skin in vivo generated by the OCT system of the present disclosure with time averaging (K=62, L=1). (with image size: 5.6 mm by 3.25 mm).

FIGS. 18A to 18H shows a comparison of field of view (FOV) between a prior art method of point scanning and the OCT system according to some embodiments.

FIGS. 19A to 19D illustrate the generation of 16 spectral bands based on Hamming Windows, with information relating to axial resolution, transverse resolution and trade-off between the two.

FIGS. 20A to 20E show various results of a comparative study of vessel visibility and noise level between a prior art point scanning and the OCT system of the present disclosure, based on the same input power and total acquisition time.

FIGS. 21A to 21D show various results of a comparative study of images obtained from a prior art point scanning system and the OCT system of the present disclosure, based on the same acquisition time, indicating an improved FOV.

FIGS. 22A to 22C show en face projection of OCT structural images of retina and overlay images taking into account motion tracking and correction.

FIGS. 23A and 23B show cross-sectional structural images in the form of amplitude frames of a human skin obtained using a prior art point scanning OCT and the OCT system of the present disclosure, indicating the axial resolution captured by the OCT system of the present disclosure being comparable to that of the prior art point-scanning.

FIGS. 24A and 24B show regions of interest for comparison of decorrelation between a prior art point-scanning (FIG. 24A) and the scanning based on OCT system 200A, 200B (FIG. 24B) were indicated. The mean decorrelation, measured from one-to-one matched vascular areas are found to be comparable between the point-scanning OCTA (0.206±0.006) and OCTA images obtained based on the system 200A, 200B (0.204±0.004) and with reference to FIG. 18H.

FIG. 25A is an intensity profile of focus along Y-axis of an OCT system of the present disclosure with specific parameters and components. FIG. 25B is a ratio plot between partial power (P) within window width (δ) for “Most Restrictive Ratio’ method plot against an arbitrary intensity.

FIG. 26A is a plot of an angular substance along Y-axis (ΔαY) against a function of wave number (K) per centimeter. FIG. 26B is a plot of a Linear error of ΔαY against the function of wave number (K) per centimeter.

FIGS. 27A to 27D show regions of interests (ROIs) on OCTA images obtained from the dependent on the number of partial spectrum decorrelation frames at each Y image position, and FIG. 27E is a plot of a speckle contrast (normalized) and decorrelation against the number of partial spectrum decorrelation frames.

FIG. 28 is a flow chart of a method for modifying an optical coherence tomography (OCT) system or device.

### DETAILED DESCRIPTION

The following detailed description refers to the accompanying drawings that show, by way of illustration, specific details and embodiments in which the disclosure may be practiced. These embodiments are described in sufficient detail to enable those skilled in the art to practice the disclosure. Other embodiments may be utilized and structural, logical changes may be made without departing from the scope of the disclosure. The various embodiments are not necessarily mutually exclusive, as some embodiments can be combined with one or more other embodiments to form new embodiments.

Embodiments described in the context of one of the systems or methods are analogously valid for the other systems or methods.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of some embodiments, the articles “a”, “an” and “the” as used with regard to a feature or element include a reference to one or more of the features or elements.

As used herein, the term “and/or” includes any and all combinations of one or more of the associated listed items.

As used herein, the scanning of a sample using the OCT system of the present disclosure is defined with respect to a sample space, with regard to a Cartesian coordinate system, wherein the depth or z-axis is always aligned with a light propagation direction, which is also referred to or known as the axial direction; and where x-axis and y-axis are the two transverse or lateral axes.

As used herein, the generation or formation of one or more OCT images are based on obtaining, deriving or retrieving an axial line profile (also referred to as A-line) using Fourier transform of a spectral interference signal. A two-dimensional (2D) cross-sectional amplitude frame, Aₙ (x,z), can be obtained by transversely scanning the sample light along the fast axis (X) radiation using a beam scanner (SC) while continuously acquiring axial (z axis or depth axis) line profiles (also referred to as A-lines). A three-dimensional (3D) image can be obtained by transversely scanning the sample light using 2-axis (fast axis x and slow axis y) scanners. If the light beam is positioned at a given y position over a sample, the amplitude frame may be mathematically expressed as:

$${A}_{n} ( x , z ) = DFT [ 2 \sqrt{{R}_{r} {R}_{s} ( z )} S ( k ) cos ( 2 k Δ p ) ] \qquad ( 1 )$$

where n is amplitude frame sequence number along the slow axis (Y), k is the free-space wave number, DFT is discrete Fourier transform with respect to 2k, z is the geometrical distance, and Rᵣ and Rₛ represent the reference reflectivity and sample reflectivity at depth z, respectively, S(k) is the source power spectral density, and Δp is the optical path delay difference between the reference and sample beams.

As used herein, the term “at least substantially” may include “exactly” and a reasonable variance.

As used herein, the term “about” or “approximately” as applied to a numeric value encompasses the exact value and a reasonable variance.

As used herein, the term “processor” refers to, or forms part of, or include an Application Specific Integrated Circuit (ASIC); an electrical/electronic circuit; a combinational logic circuit; a field programmable gate array (FPGA); a computer sever (shared, dedicated, or group) that executes code; other suitable hardware components that provide the described functionality; or a combination of some or all of the above, such as in a system-on-chip. The term processor may include memory (shared, dedicated, or group) that stores code executed by the processor.

In the following, embodiments will be described in detail.

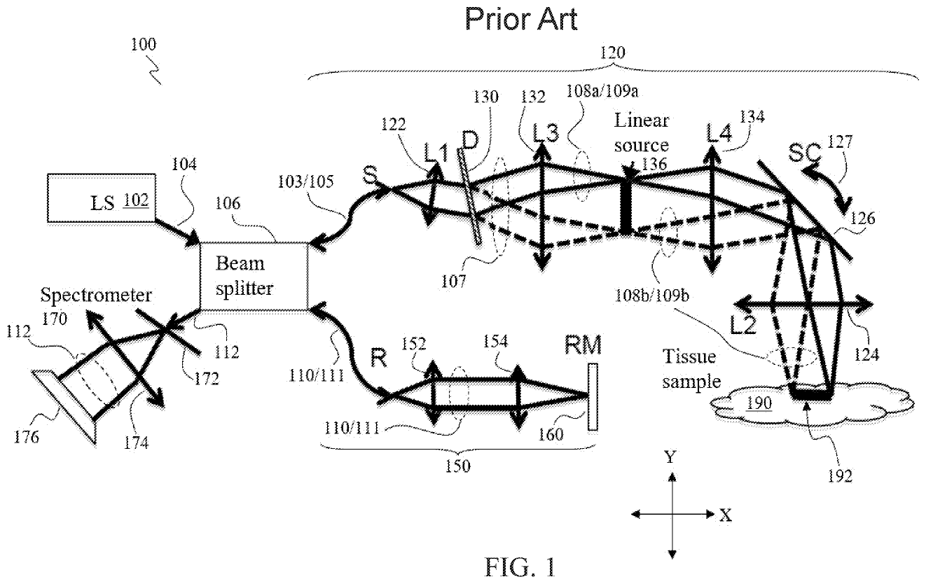

FIG. 1 shows a prior art optical imaging system 100. The optical imaging device 100 includes a light source (LS) 102 which may provide a light 104, for example in the form of a small-source, to a beam splitter 106. The beam splitter 106 may split the light 104 into two 103, 110, which are respectively provided to a sample arm(S) 120 and a reference arm (R) 150 of the optical imaging system 100. Light 103 may be collimated by a collimation lens (L1) 122, which then passes through a dispersive element (D) 130. The dispersive element (D) 130 may include prism(s), grating(s) or other dispersive component(s). The dispersive element (D) 130 may spread the radiation or light spectrum of the small source (light 103) to an extended-source, e.g. a line or linear source 136. In details, the dispersive element (D) 130 may generate an extended-source illumination pattern 107 from the light 103. As a result of passing through the dispersive element (D) 130, spectral bands making up the extended-source illumination pattern 107 may become separate from each other. As shown in FIG. 1, the illustrated extreme spectral bands 108a, 108b are separate from each other. A relay optics assembly having relay optics lens (L3 and L4) may be provided such that the lens (L3) 132 may focus the plurality of spectral bands, including spectral bands 108a, 108b, to form an intermediate or apparent linear source 136, which is then collimated by the lens (L4) 134. The spectral bands, including bands 108a, 108b, may then be directed by a beam scanner or a scanning device (SC) 126 towards a sample (e.g. a tissue sample) 190 to be imaged, forming a line illumination 192 at the sample 190. Light radiation at a given point within the line 136 may have an arrow spectral line width. The centre wavelength and the line width at a given point in the linear source 136 may be determined by the dispersive property of the dispersive element (D) 130 and the focusing power of the relay lens (L3) 132. The linear source 136 may be located at a plane conjugated with the sample 190, so that on the sample 190, the illumination light radiation field may also be a line.

An objective lens (L2) 124 may be arranged to focus the extended-source illumination pattern 107 including the spectral bands towards a focal plane on the sample 190. Respective spectral bands of the extended-source illumination pattern 107 may illuminate respective sections of the sample along the line illumination 192. The scanning device (SC) 126 may be moved, for example in directions represented by the arrow 127, during the scanning process so as to scan different parts of the sample 190 so that a two or three-dimensional image of the sample 190 may be formed.

Interaction between respective spectral bands and the respective sample sections result in respective return lights being generated. Each return light may include light reflected and/or light scattered from the sample section. Respective return lights, for example 109a, 109b, may propagate through at least substantially similar optical paths as for the respective spectral bands, but in an opposite direction, through the objective lens (L2) 124, the scanning device (SC) 126, the relay optics lens (L4) 134 and (L3) 132, the dispersive element (D) 130 and the lens (L1) 122, towards the beam splitter 106 to define a sample light 105.

In the reference arm (R) 150, light 110 may propagate through a pair of lenses, for example a collimation lens 152 which may collimate the light 110, and a focusing lens 154 which may then focus the collimated light onto a reference mirror (RM) 160. Light 110 incident on the reference mirror 160 is reflected by the reference mirror 160, which then propagates through the collimation lens 152 and the focusing lens 154 towards the beam splitter 106 to define a reference light 111.

At the beam splitter 106, the sample light 105 and the reference light 111 may interfere with each other or may be combined to form an interference signal 112 to be received by a spectrometer 170 acting as a detector. The spectrometer 170 may include a grating 172 to spectrally disperse the interference signal 112, which is then collimated by a collimation lens 174 prior to being detected or captured by a detecting element 176, e.g. a camera. Processing may be carried out to obtain spectral information corresponding to the sample 190 illuminated by the extended-source illumination pattern 107 from the interference signal 112.

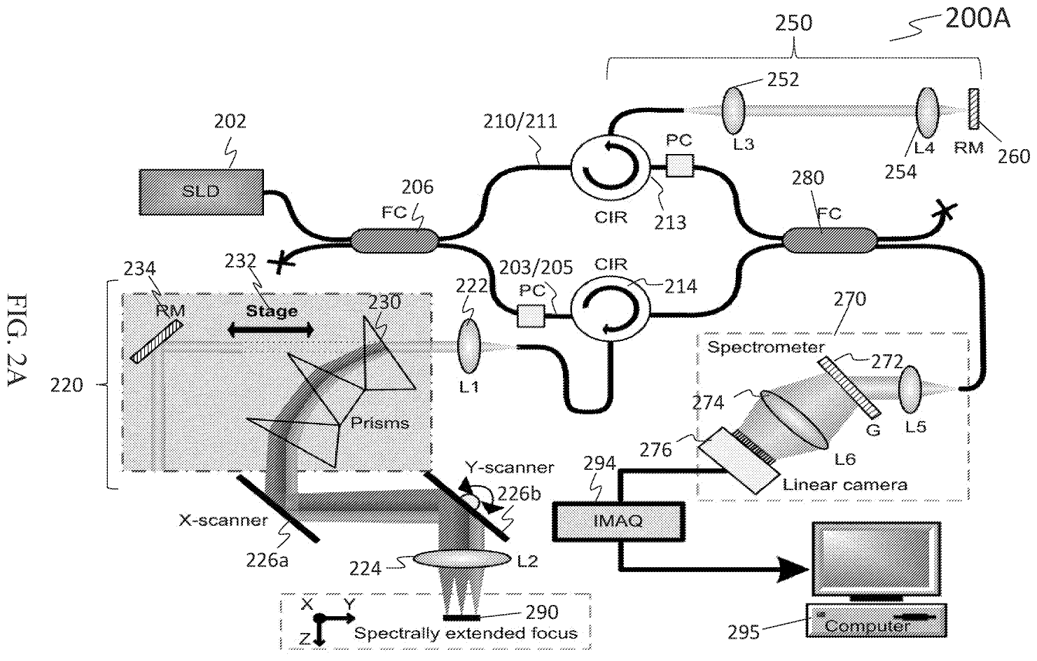

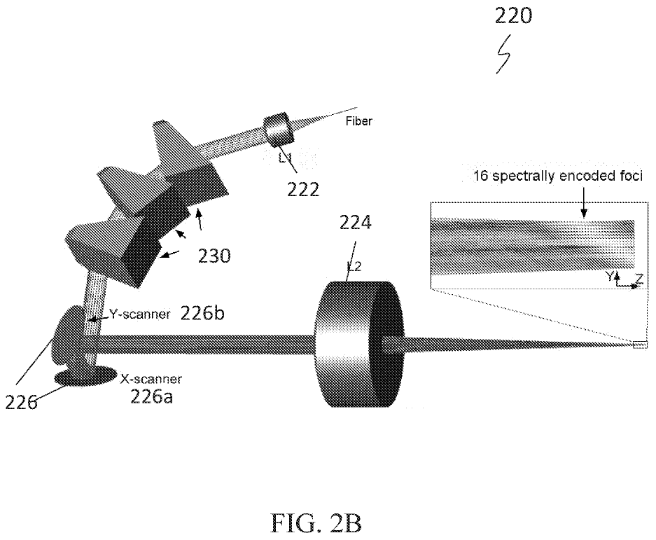

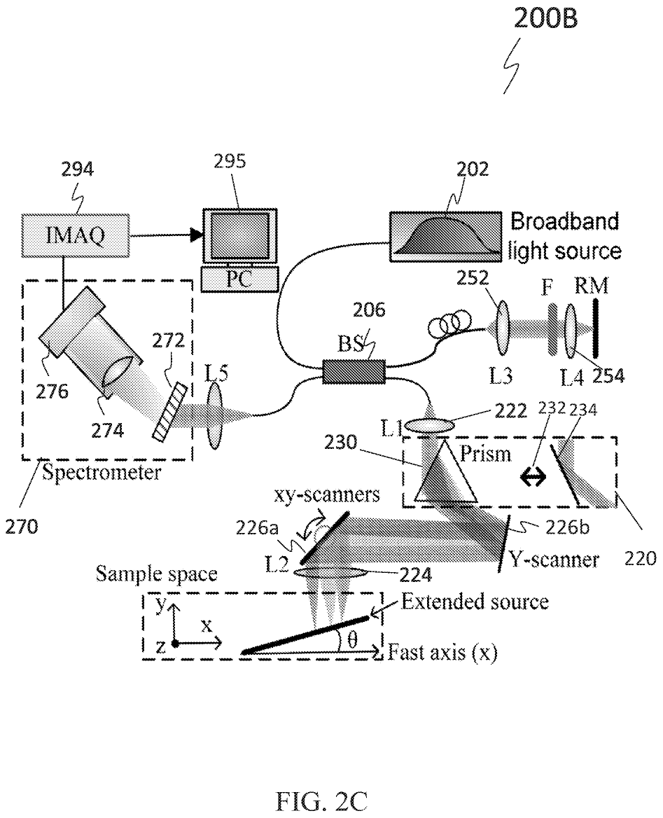

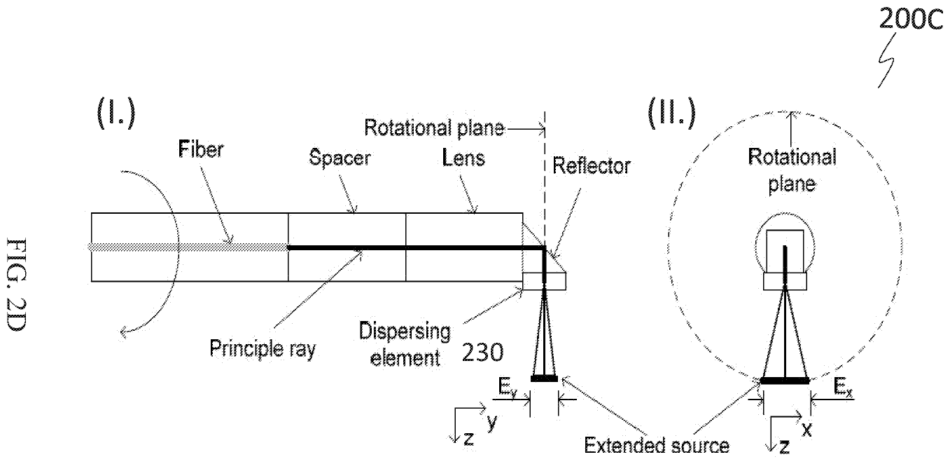

FIG. 2A shows a schematic view of an optical imaging system 200A, according to various embodiments of the present disclosure. The various elements are similar to FIG. 1 but the sample arm 120 is replaced with the optical setup 220 as elaborated further with reference to FIG. 2B and FIG. 2C. The optical setup 220 is configured to direct light to a sample, such as, but not limited to, a mammalian tissue (e.g. a human retina or skin tissue).

In the embodiment shown in FIG. 2A, the output of the light source 202, which may be in the form of one or more superluminescent diodes, is split by a beam splitter/fiber coupler 206 to the reference light 211 and the sample light 205. The reference light 211 is guided to a reference arm optics 250 via either a fiber or free space optics, which may be in the form of a fiber circulator 213. The reference arm optics 250 comprises a collimation lens 252 which the reference light 211 is incident on, which is subsequently focused onto a reference reflector (RM) 260 by lens (L4) 254. The sample light 205 is guided through the sample arm optics 220 to the sample 290, which may be, for example, a tissue sample forming part of a human eye or a human hand. The sample arm optics 220 comprises a dispersive element 230 to generate an extended source for illuminating the sample 290. Light 203 may be guided to the optical setup (sample arm) 220 via either a fiber or free space optics, which may be in the form of a fiber circulator 214. The respective return lights may propagate through at least substantially similar optical paths to define a sample light 205.

The dispersive light back-reflected from the reference arm 250 and back-reflected or backscattered from the sample arm 220 are combined by another beam splitter or fiber coupler 280 and part of the interference signal is directed to a detection arm, which may be in the form of a spectrometer 270. The spectrometer 270 may include a grating 272 to spectrally disperse the interference signal, which is then collimated by a collimation lens 274 prior to being detected or captured by a detecting element 276, e.g. a linear camera. Processing may be carried out to obtain spectral information corresponding to the sample 290 illuminated by the extended-source illumination pattern from the interference signal.

The spectral interference data may be transferred and/or stored, further processed in a computer processor via image acquisition electronics (IMAQ) 294.

In the embodiment shown in FIG. 2A, the dispersive element is orientable such that the extended source is disposed at a non-zero angle with respect to a fast axis. In some embodiments, the non-zero angle is an acute angle. In some embodiments, the non-zero angle is a right angle about or substantially 90 degrees.

In the sample arm 220, one or more dispersive elements 230, such as a prism or grating, disperses the light collimated by lens (L1) 222 into multiple spectral bands. Each of the spectral bands may follow a distinct propagation direction as the result of chromatic diffraction, so that at the focal plane of the sample, a linear, extended source is created.

Objective lens (L2) 224 may be arranged to focus the extended-source illumination pattern including the spectral bands towards a focal plane on the sample 290. Respective spectral bands of the extended-source illumination pattern may illuminate respective sections of the sample along the line illumination.

In the sample arm 220, a beam scanner or a scanning device (SC) 226 comprises a x-scanner (for scanning along fast axis) 226a and y-scanner (for scanning along slow axis) 226b. The x-scanner 226a is configured to scan a plurality of times along the fast axis at each of a respective plurality of positions along the slow axis. This may be referred to as repeated-scans-at-the-same-y-position (RBSSYP). The x-scanner 226a and y-scanner 226b may be adjusted (via rotation, translation or both) during the scanning process so as to scan different parts of the sample 290 so that a two or three-dimensional image of the sample 290 may be formed. In some embodiments, the scanning device 226 may include one or more optical scanners, such as Galvanometer scanners. In some embodiments, the sample arm 220 may comprises a translation stage 232 operable to switch between a point scanning mode/scheme utilizing a reference mirror 234, and the extended source scanning mode/scheme utilizing the dispersive element 230 and the set up shown in FIG. 2B.

In the optical imaging system 200A, the IMAQ 294 may send the acquired image to a computer 295 for processing. A plurality of partial-spectrum frames, including at least one OCT and/or at least one OCTA image from the partial-spectrum frames may be generated by the processor 295.

FIG. 2B shows the three-dimensional close-up schematic view of sample arm optics 220 comprising collimated lens (L1) 222, light dispersive elements 230 and the positioning of the x-scanner 226a at about 90 degrees from the extended source.

FIG. 2C shows another embodiment of the OCT system 200B with similar working principles. In the embodiment shown in FIG. 2C, the light source 202 is depicted as a generic broadband light source, and a sample space is shown wherein the extended source is disposed at a non-zero angle to the fast axis.

In some embodiments, one or more polarization controllers may be arranged with the fiber circulators 213 and 214 to allow polarization of the sample light and reference light.

Another embodiment in the form of an endoscopic probe 200C is shown in FIG. 2D. In the embodiment shown in FIG. 2D, a dispersive element 230 may be added to a conventional endoscopic probe, at its distal end optics, which can be either after a reflector (shown in FIG. 2D(i.)—a side schematic view of the endoscopic probe) or between the reflector and the lens. The dispersing element 230 may be operable to generate a number of spectral bands, which propagate along distinct directions. By doing so, the illumination line forms an extended source that can be illuminated on a sample to obtain images thereof. The rotational plane of the probe is the plane formed by rotating the monochromatic beam with a centre wavelength of the input light (as shown in FIG. 2D(ii.)—a front schematic view of the endoscopic probe). As described in other embodiments, the angle between the extended source and the rotational plane is 0<θ≤90°. The terms E_{y} and Eₓ are defined and elaborated in paragraph [0065].

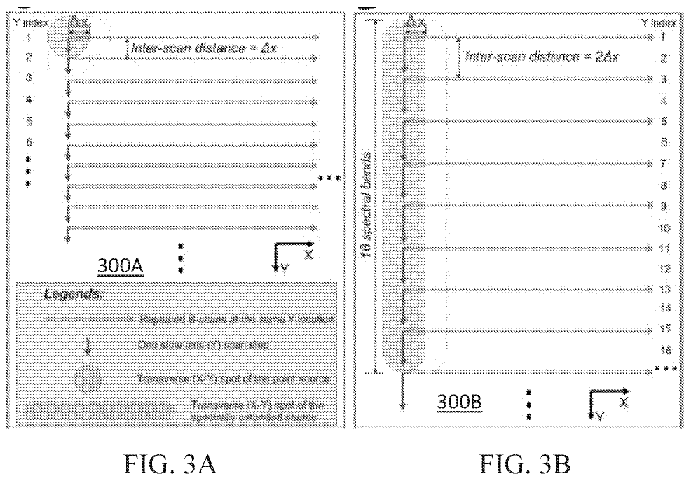

FIG. 3A illustrates a scanning pattern 300A of a point source OCT compared to an exemplary scanning pattern 300B using the extended source scanning of the system 200A or 200B. In relation to the standard point source OCT, the transverse point-spread function (PSF) is circular and its transverse size is determined by diffraction theory. The X and Y inter-scan distance, Δx and Δy, respectively, is equal or less than half of full-width at half maximum (FWHM) of the transverse PSF. For the scanning pattern based on the point source OCT, the transverse PSF is a line along a transverse direction (e.g. Y direction), and by splitting the light source spectrum into P bands with each band center at a distinctive transverse position of the sample 290. Different from the case of the standard point-scanning OCT, where the inter-scan distance Δx=Δy, in the system 200A or 200B, the inter-scan distance along the Y-axis is 1≤L≤P times of that of X axis, Δx, that is, each of the respective plurality of positions along the slow axis is set at an inter-scan distance L of 1≤L≤P, wherein P is the number of partial-spectrum frame(s). In this regard, L<P indicates there are overlap along the Y-axis between the illuminated areas of RBSSYP executed at adjacent Y positions. For example, in FIG. 3B, the number of shown spectral bands is 16 and the center-to-center distance between adjacent bands along the Y-axis is Δx. With L=2, the overlap length along the Y-axis between adjacent B scans is 14*Δx. One can perform DFT on each of 16 partial-spectrum interference data acquired over a B-scan to generate 16 partial-spectrum amplitude frames. If N=2, there will be two repeated B-scans at each Y position, and 2 APC, amplitude, phase or complex (APC) variation frames are generated to produce 1 partial-spectrum APC variation image.

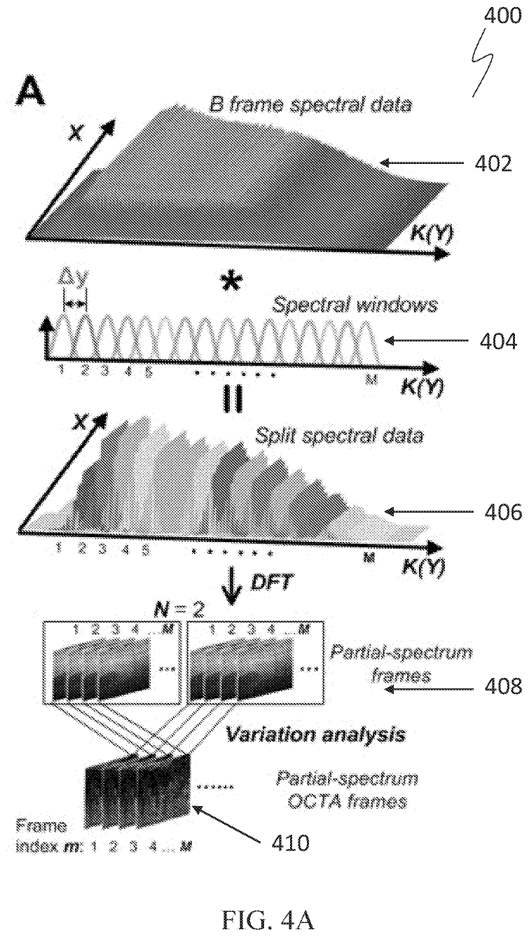

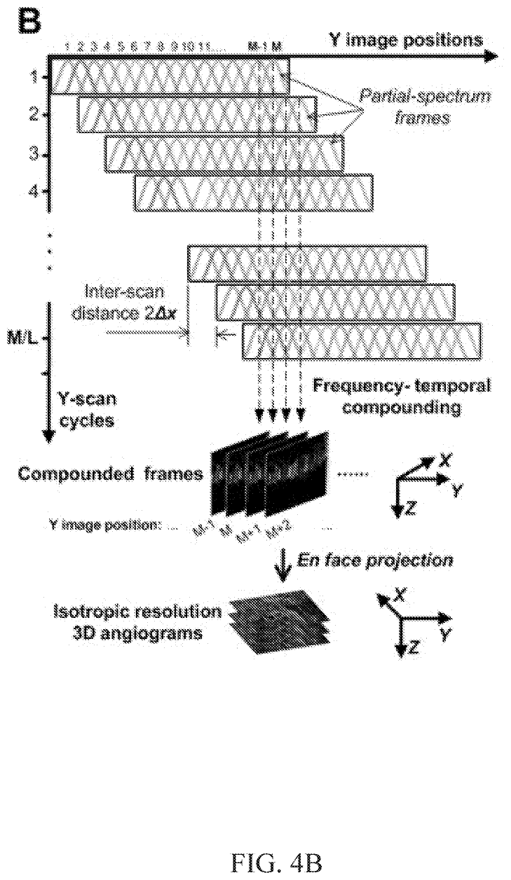

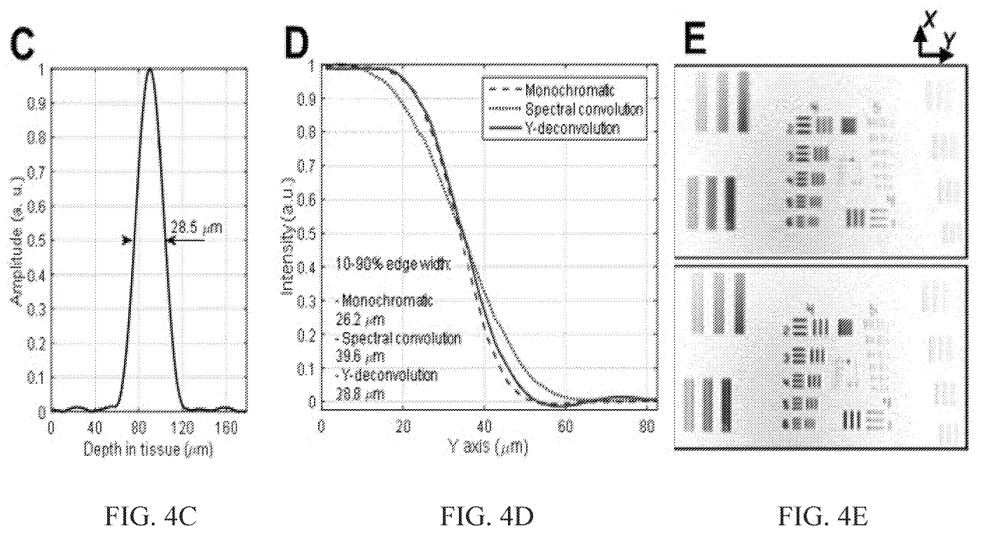

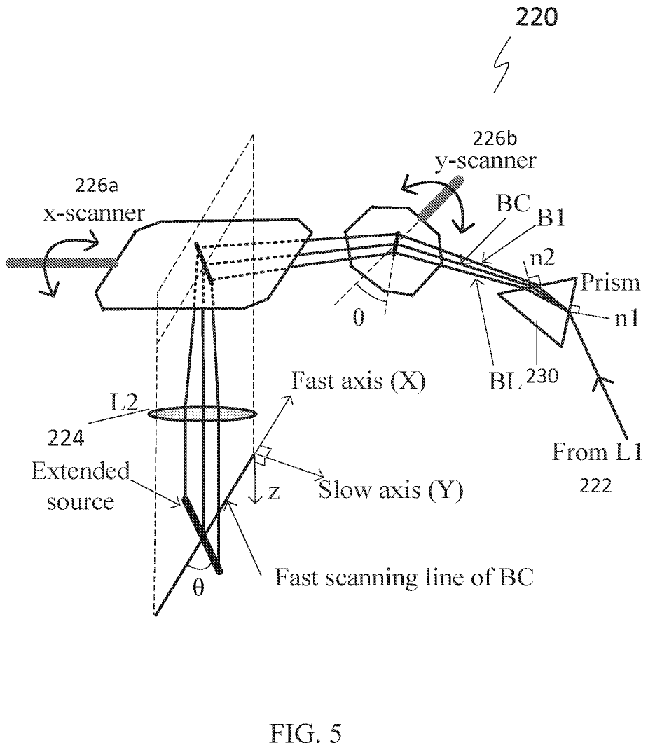

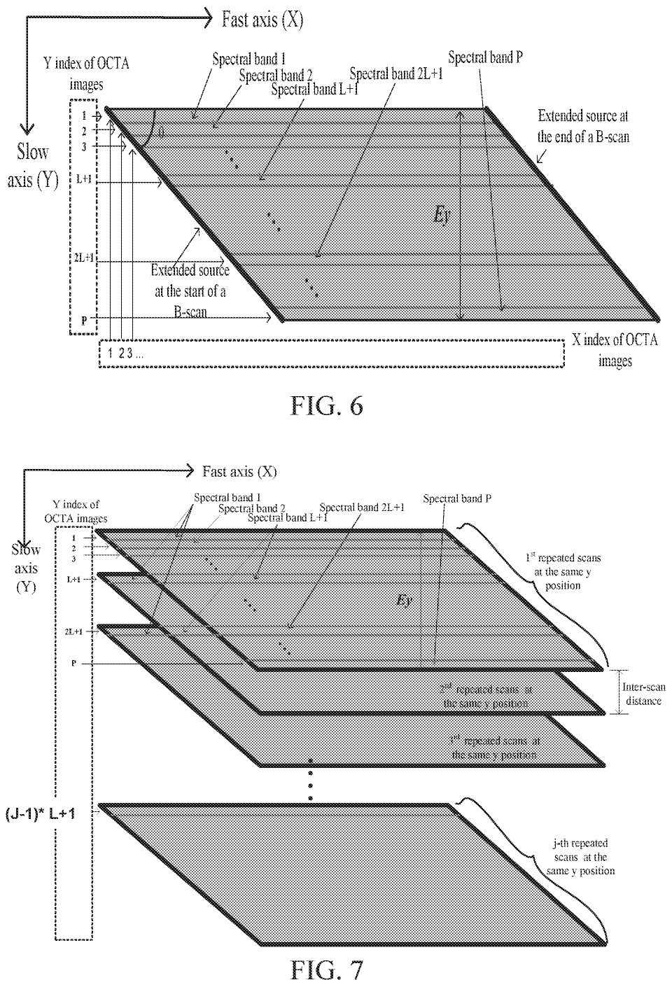

FIG. 4A illustrates a process 400 to generate partial-spectrum APC variation frames or images based on an angiogram application. A spectral interferogram comprising optical pulse information/data is split into P number of partial spectral interference data 406 by multiplying the full spectral interferogram 402 with spectral windows, which are Gaussian spectral band-pass filters 404 equally spaced in a k space or Y axis. Each split P partial spectral interference data 406 then undergoes a Discrete Fourier Transform (DFT) process. P axial profiles or partial spectrum frames 408, each at distinct transverse position can be obtained from the DFT process, in contrast to one axial profile in the case of the standard point-source OCT. Variation analysis may then be carried out to obtain partial spectrum OCTA frames 410.

FIG. 4B shows a process wherein the partial-spectrum frames 408 undergo a temporal averaging of the partial-spectrum frames for each of the respective plurality of positions to generate a plurality of respective OCT images associated with the respective plurality of positions. In particular, the processor 295 may be configured to perform frequency compounding of the partial-spectrum frames for each of the respective plurality of positions. All partial-spectrum (APC) frames located at the same transverse position are averaged into a compounded frame, which is essentially a frequency compounding process.

It is contemplated that amplitude deconvolution is suited as a schematic for APC variation. In some embodiments, the partial-spectrum decorrelation images generated at the same Y position is M=8 as illustrated in FIG. 4B. In the embodiment shown in FIG. 4B, eight (8) partial-spectrum decorrelation frames are averaged to a compounded decorrelation frame. As the Y-transverse PSF can be modelled as the convolution of monochromatic PSF with the spectral window, a deconvolution along Y axis may be performed to restore the transverse resolution. As shown in FIGS. 4C to 4E, the 3D spatial resolutions are measured to be isotropic. FIG. 4C is a plot of measured axial resolution based on an amplitude (in arbitrary units a.u.) vs depth in tissue (in micrometer μm), FIG. 4D is a plot of a 10-90% edge width of the monochromatic beam based on an intensity (in a.u.) vs Y-axis (in μm), spectral-window convoluted beam, and deconvolution result. FIG. 4E shows a US Air Force 1951 Resolution chart images before (top) and after (bottom) deconvolution.

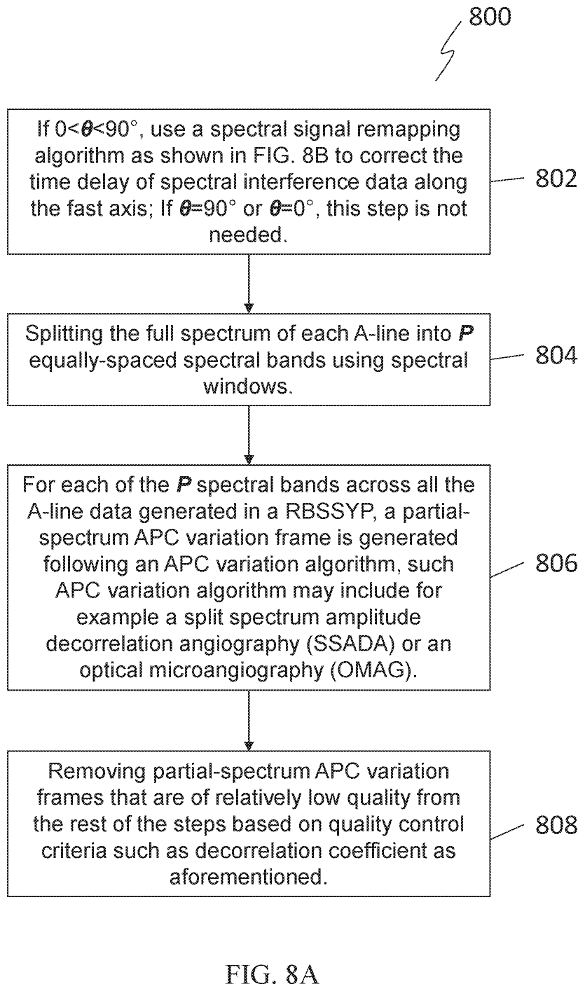

FIG. 5 shows an embodiment of the sample arm 220 wherein the dispersive element 230 is orientable such that the extended source is disposed at an acute angle θ, that is, where 0<θ<90° to achieve extended source scanning. When in use, light collimated by lens (L1) 222 propagates in the plane that may be defined or determined by normal vectors of two facets of the prism: n1 and n2. The plane may be referred to and is denoted as an n1−n2 plane. The output of the dispersive element 230, such as a prism, are multiple beams of distinct spectral bands, which all propagate in the n1−n2 plane. In FIG. 5, the beams labelled B1, BC, and BL represent the principle ray(s) of the collimated beam of λ1, λc and λL, respectively, where λ1, λc and λL are short cut-off wavelength, center wavelength, and long cut-off wavelength of the broadband source spectrum, respectively. The angle θ can be adjusted by rotating n1-n2 plane with the principle ray BC as the rotational axis, relative to the x and y scanner rotational axes. In the focal plane of the objective lens (L2) 224, the angle θ is the angle between the extended source and the x-axis or fast scanning line of any monochromatic light beam.

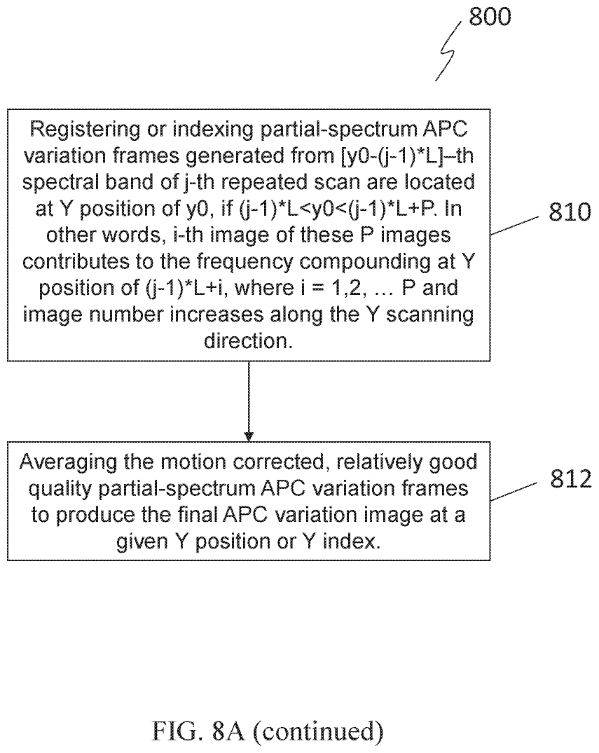

In some embodiments where a length of the extended source is denoted E, a component along the x-axis will be denoted Eₓ=E*cos θ, and a component along the y-axis E_{y}=E*sin θ. The spectrum of each amplitude frame may cover a range of E_{y} along the Y axis, where E_{y} is multiple (P) of half transverse resolution (FWHM) of OCT. By dividing the illumination spectrum into P equally spaced spectral bands, a P number of partial-spectrum APC variation frames from spectral interference data acquired in one B-scan may be obtained as illustrated in FIG. 6. In other words, P partial-spectrum APC variation frames can be obtained from data acquired in one RBSSYP. Each of P partial-spectrum frames may be centered at a distinct y position, and the center-to-center distance between each of the adjacent frames along Y is Δx.

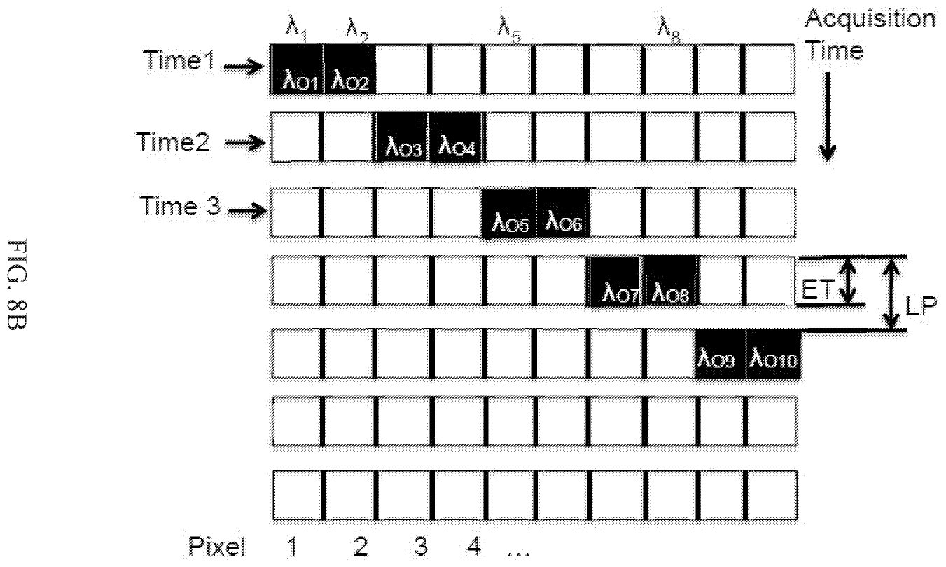

In some embodiments, the scanner 226 may be configured such that an inter-scan distance can be set to be larger than Δx (L>1) as illustrated in FIG. 7. In such a configuration, scanning may be advantageously performed L times as fast as the conventional point source OCT. If L is configured to be equals to P, there will be no overlap between two adjacent RBSSYP along the y-axis as shown in FIG. 6. In embodiments where the scanner 226 is configured such that P>L, there will be significant overlap between consecutive RBSSYP as shown in FIG. 7.

In some embodiments, to produce the compounded APC variation image at a given X or Y position, partial-spectrum APC variation frames located at the X or Y position acquired at all times may be averaged based on temporal averaging as well as frequency compounding. For example, as shown in FIG. 7, for Y index of L+1, the partial-spectrum APC variation frame generated from the spectral band L+1 of the 1st RBSSYP and the APC variation frame generated from the spectral band 1 of the 2nd RBSSYP are averaged. In another example, for Y index of 2L+1, the partial-spectrum APC frame generated from the spectral band 2L+1 of the 1st RBSSYP, the partial-spectrum APC frame generated from the spectral band L+1 of the 2nd RBSSYP, and the partial-spectrum APC frame generated from the spectral band 1 of the 3rd RBSSYP may be averaged.

In some embodiments, the processor 295 is configured to check for at least one low quality frame of the partial-spectrum frames and remove the at least one low quality frame prior to generating the partial-spectrum APC frames.

In an example, motion during repeated scans at a same Y location may cause motion artifacts in partial-spectrum APC frames, and partial-spectrum APC frames that are of relatively low quality may be removed before temporal averaging. A relatively simple way to determine the quality of APC frames is to evaluate the mean decorrelation coefficient in non-vascular areas, for example epithelium. Low quality OCTA frames may have high decorrelation coefficient in non-vascular areas, and it is less likely that all the partial-spectrum OCTA frames at a y position are significantly degraded by motion. Therefore, rejecting a few partial-spectrum OCTA frames with the highest mean decorrelation coefficient in non-vascular areas may significantly improve the resultant image set quality.

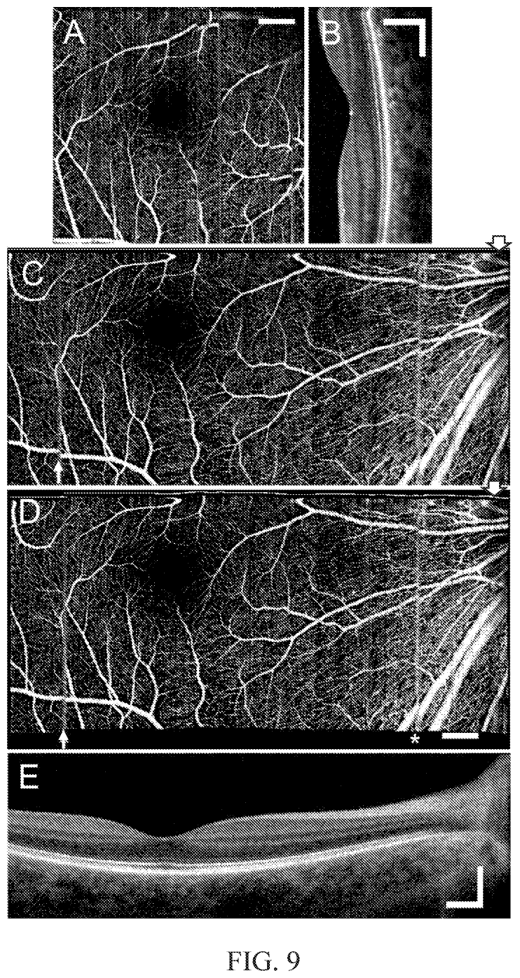

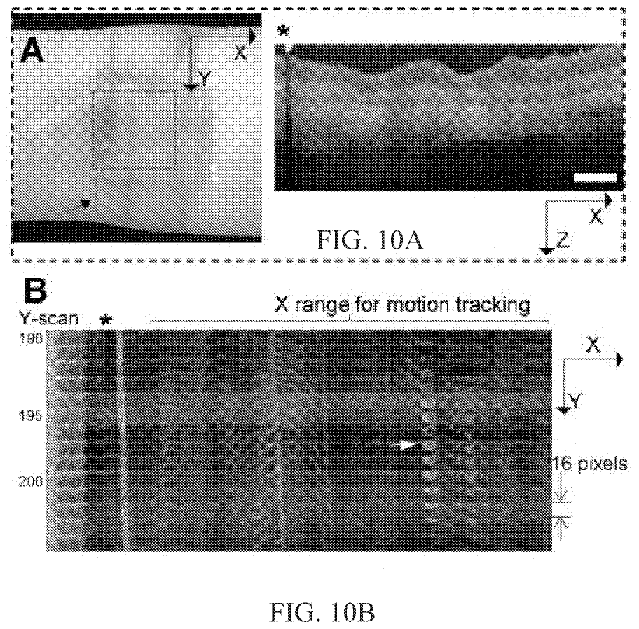

FIG. 8A shows a flow chart depicting a process 800 for processing the scanned data collected at the IMAQ 294 by the processor 295, comprising the following steps.

Step 802-If 0<θ<90°, use a spectral signal remapping algorithm as shown in FIG. 8B to correct the time delay of spectral interference data along the fast axis; If θ=90° or θ=0°, this step is not needed.

Step 804—Splitting the full spectrum of each A-line into P equally-spaced spectral bands using spectral windows.

Step 806—For each of the P spectral bands across all the A-line data generated in a RBSSYP, a partial-spectrum APC variation frame is generated following an APC variation algorithm, such APC variation algorithm may include for example a split spectrum amplitude decorrelation angiography (SSADA) or an optical microangiography (OMAG).

Step 808—Removing partial-spectrum APC variation frames that are of relatively low quality from the rest of the steps based on quality control criteria such as decorrelation coefficient as aforementioned.

Step 810—registering or indexing partial-spectrum APC variation frames generated from [y₀−(j−1)*L]-th spectral band of j-th repeated scan are located at Y position of y₀, if (j−1)*L<y₀<(j−1)*L+P. In other words, i-th image of these P images contributes to the frequency compounding at Y position of (j−1)*L+i, where i=1, 2, . . . P and image number increases along the Y scanning direction.

Step 812—Averaging the motion corrected, relatively good quality partial-spectrum APC variation frames to produce the final APC variation image at a given Y position or Y index.

FIG. 8B shows a spectral signal remapping algorithm. The spectral data acquired by the spectrometer 270 is remapped according to the calculated arrival time of each wavelength to obtain the entire interference spectrum of a point O. The spectral data is acquired as a function of time (Time 1, 2, 3, . . . ). Each horizontal array represents a spectrum acquired at a given time using the spectrometer 270. Each cell of a horizontal array represents the data from each pixel of the line camera 276 of the spectrometer 270, and black cells represent the spectral data originated from the point O. ET refers to exposure time, and LP refers to camera acquisition line period.

It is appreciable that in FIG. 8B, the different spectral bands arrive at a reference point O as a function of time. Spectral signal acquired by the spectrometer for the point O may be remapped in time domain to obtain the correct axial profile. With reference to point O, the exact arrival time of each wavelength may be calculated based on the parameters of the diffraction element 230, the relay optics 253, 254 and the beam scanner 226 angular position. The spectral data acquired by the spectrometer 270 is remapped according to the calculated arrival time of each wavelength to obtain the entire interference spectrum of point O. For example, at time 1 the spectral band with centre wavelength λ_{O1} is dwelling right at point O, so that the first pixel of the spectral data acquired at time 1 is the first pixel of the spectral data for point O; for the wavelength 202, the calculated arrival time is between two acquisitions at time 1 and time 2, the data of point O can be obtained through interpolating adjacent spectral data in time domain. FIG. 8B exemplifies the case of nearest-point interpolation.

The system 200A and method 800 will next be described in the context of an application in ophthalmology (particularly angiography) to provide a detailed visualization of, for example, one or more vascular networks in an eye sample.

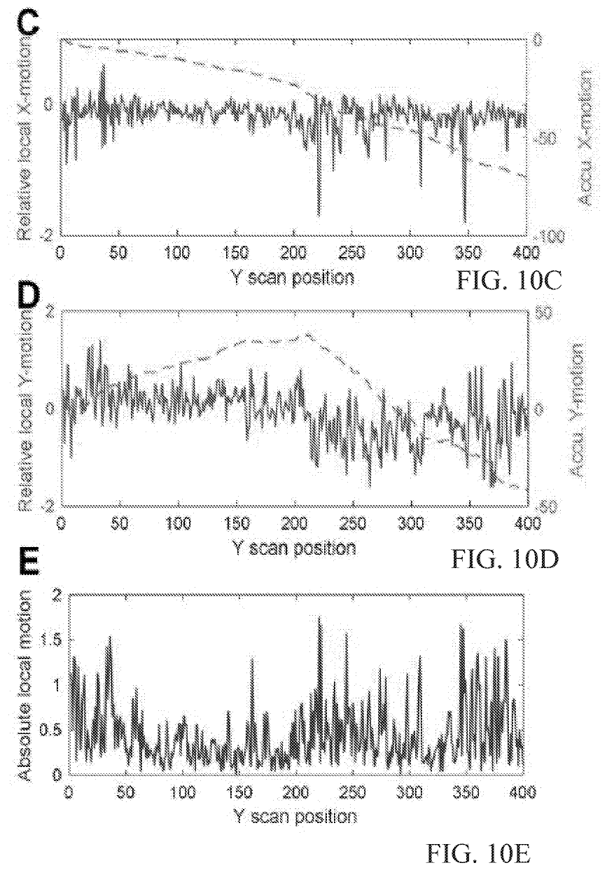

Results using system 200A and method 800 using the configuration of the sample arm 220 are shown in FIGS. 9 to 14 to demonstrate the effectiveness of the system and method. Comparative studies are made with respect to conventional point source scanning, where applicable.

Field of view improvement: Retinal angiograms were acquired with 67,590 Hz A-line rates for both the point scanning and the extended source scheme with the same data size: 400 A-lines per B-frame, 400 Y-scan positions, and N=2 repeated B-scans per Y-scan position. For images acquired with the point-scanning scheme, the field of view (FOV) is 3.44×3.44 millimeters (mm). For images generated with the system 200A, FOV is 3.44×6.88 mm (width×height). With an estimated lateral resolution of ˜16.3 μm at retina, the sampling density for both scheme is close to Nyquist sampling requirement. For retinal angiograms, the whole spectrum was split into M=8 equally spaced bands in wavenumber domain. The inter-scan distance L was set to 2 so that the field of view obtained by system 200A (see FIGS. 9C and 9D) is twice as large as that of the point source (see FIG. 9A). FIG. 9B is an OCT image obtained based on a prior art point-scanning for comparison with FIG. 9E, which is a corresponding OCT image obtained based on the system 200A, 200B of the present disclosure.

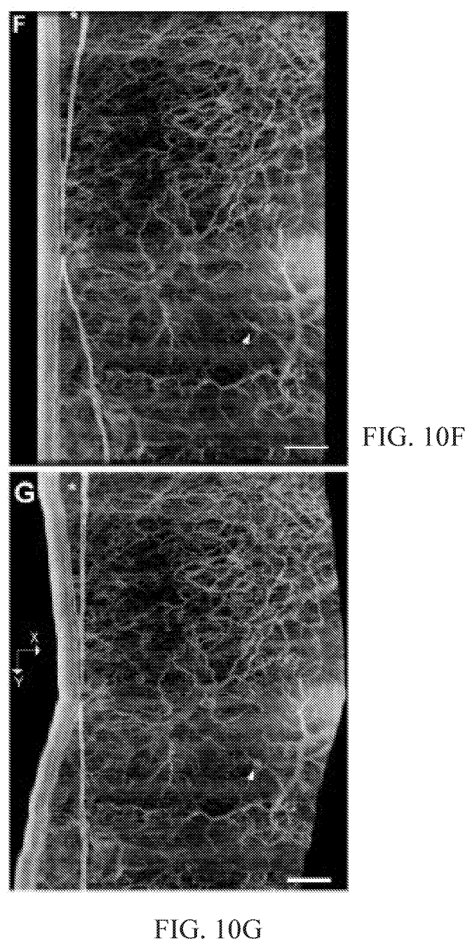

Motion tracking: The system 200A, 200B of the present disclosure performs X-Z or Y-Z priority scanning so that a three-dimensional (3D) dataset may be generated from spectral interference data acquired in one B-scan. In some embodiments, the corresponding OCTA dataset may have a size of 512 (X) by 16 (Y) by 1024 (Z), where 512 is the Aline number per B-scan, 16 is number of spectral bands P, and 1024 is half of DFT length. As there are 14 pixels overlap in Y axis between 2 adjacent B-scan data, the relative motion between adjacent B scan data may be calculated and corrected by one of the existing motion correction algorithms, such as 2D (XY) correlation. FIG. 10B shows 14 en face (XY plane) images, and each of them is the result of Z projection of OCTA data generated from 2 RBSSYP. The numbers on the left is Y frame index. The adjacent en face images have 14 pixels in common (along Y axis) if there is no motion in-between, as can be clearly seen with images of an air bubble (see arrow). FIGS. 10C and 10D show the local and accumulative motion along X and Y direction respectively, measured by a two-dimensional (2D) correlation method. FIG. 10A shows a thin and straight metal wire attached to skin surface, as shown in an arrow in the left side photo and a star in the cross-sectional OCT intensity image (right) as a motion reference. The skin was intentionally moved during the image acquisition in both X and Y directions, so that the shadow of the metal wire (star) in the en face (XY) plane is distorted as shown in FIG. 10E. The motion tracking excludes the part of image containing the shadow of the metal wire shown in FIG. 10B. The motion correction eliminated the motion artifacts in both X and Y directions as shown in FIG. 10G. It may be noted that the Y distance error caused by motion between the two vessel bifurcations, indicated by arrowheads in FIGS. 10F and 10G, are also corrected. FIG. 10E shows an absolution local motion calculated as square root of sum of relative local motions squared.

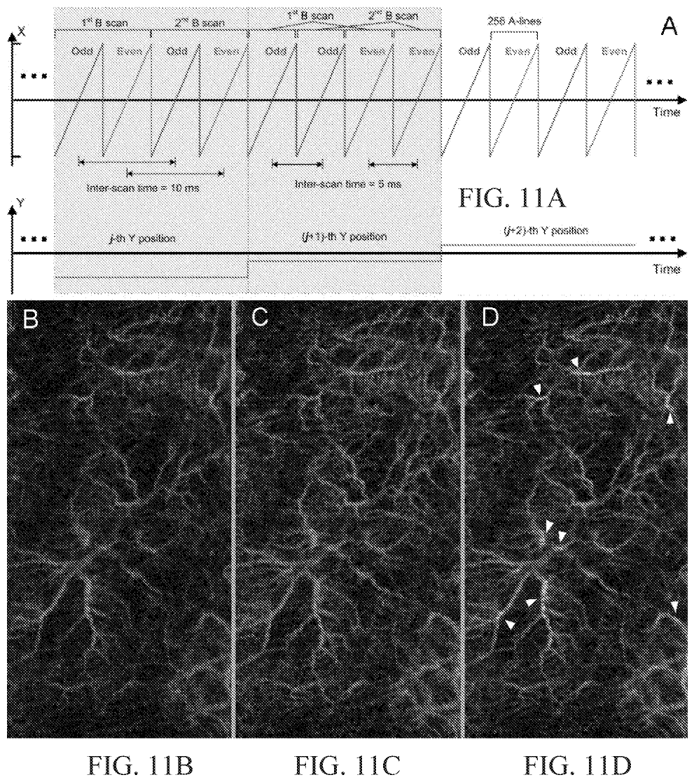

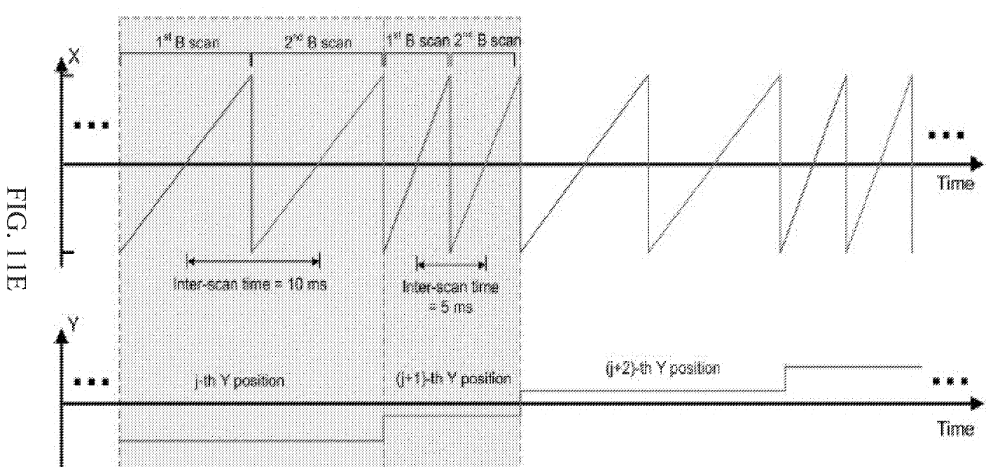

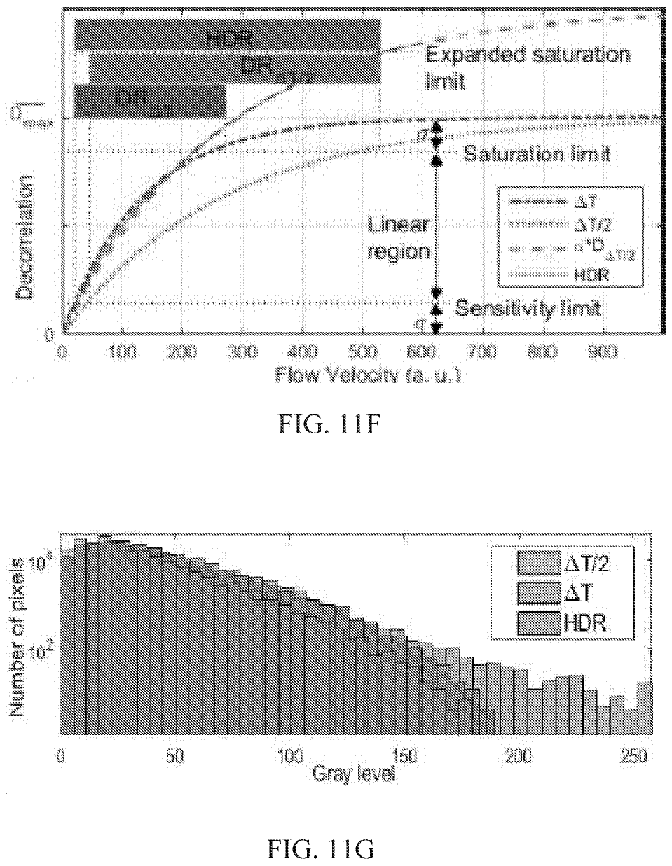

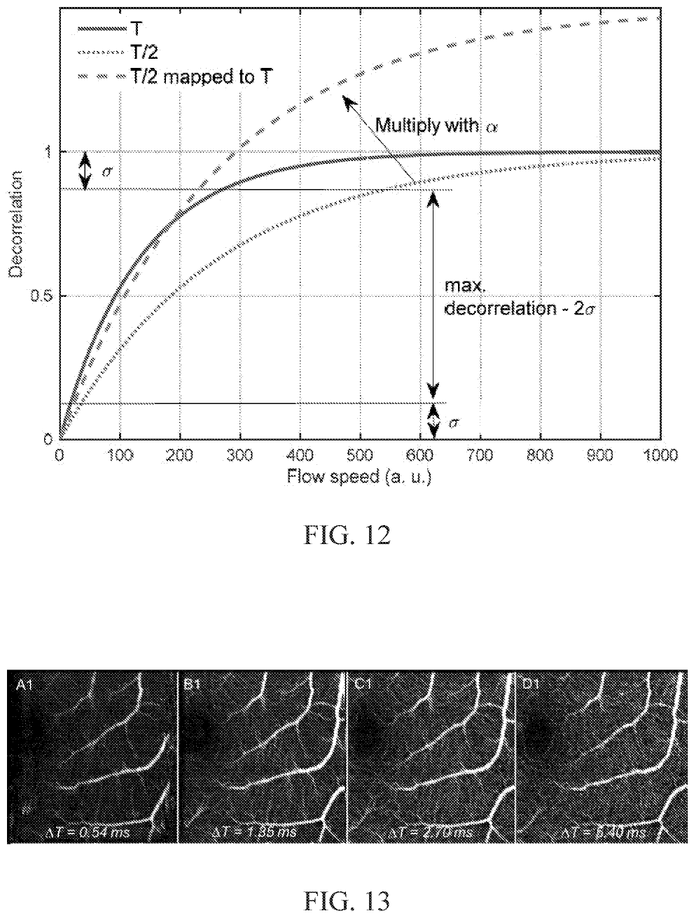

Variable or multiple inter-scan time with N=2 In some embodiments, the processor 295 may be configured to effect multiple or dynamic inter-scan time. One possible way to achieve multiple inter-scan time is to use different A-line period for B scans at different Y position as illustrated in FIG. 11. In FIG. 11, when the beam dwells at the j-th Y position, there are 2 repeated B scans executed by the fast (X-axis) scanner. Assuming the A-line number per B scan is 512 and line period is 19.53 micro-seconds (μs), the inter-scan time is calculated to be 512×19.53 μs=10 milli-seconds (ms). When the beam dwells at the (j+1)-th Y position, 2 repeated B scans may be executed by the fast (X) scanner. In this case, the A-line number per B scan is still 512 but the line period is 9.77 μs, the inter-scan time is 512×9.77 μs=5 ms. In the next Y position (J+2), the A-line period changes back to 19.53 μs. Therefore, alternating A-line period along Y positions can achieve at least 2 different inter-scan time.

It is contemplated that alternating A-line period along Y positions may minimize or negate the introduction of any image artifact. For example, according to FIG. 4B, from the data acquired in each RBSSYP P=16 partial-spectrum APC variation images are generated, and i-th image of these 16 images contributes to the frequency compounding at Y position of (j−1)*L+i, where I=1, 2 . . . . P and image number increases along the Y scanning direction. Therefore, at each Y position there are P/L=8 images to be averaged for frequency compounding, including 4 images of inter-scan time=5 ms and 4 images inter-scan time=10 ms.

In addition, frequency compounding can be done such that two 3D datasets are generated: one generated by averaging partial-spectrum frames of inter-scan time=5 ms and the other generated by averaging those of inter-scan time=10 ms. This can be simply achieved by averaging 4 partial frames of the same inter-scan time instead of 8 at each Y position.

Besides using different A-line period for B scans at different Y position, another way to achieve multiple inter-scan time is to split a full B-scan into multiple partial B-scans, for example, two half B-scans comprising of a B-scan of odd X positions, and a B-scan of even X positions (FIG. 11A), and the processor 295 can be configured as such. When the beam dwells at the j-th Y position, there are 2 repeated full B scans executed by the fast (X) scanner, and each is composed of a scan dwells only at all the odd X positions and another scan dwells only at all the even X positions. Assuming the A-line number per full B scan is 512 and line period is 19.53 μs, the inter-scan time between 2 odd scan or 2 even scans is 512×19.53 μs=10 ms. When the beam dwells at the (j+1)-th Y position, there are 2 repeated full B-scans executed by the fast (X) scanner. The A-line number per B scan is still 512 and the line period is still 19.53 μs μs, the inter-scan time between 2 odd scan or 2 even scans is 256×19.53 μs=5 ms. At each Y position, the 1^{st} full B-scan and 2^{nd} full B-scan can be obtained by combining the corresponding odd and even scans. FIGS. 11B to 11D demonstrate the benefit of the 2 inter-scan time. In the image generated by data of inter-scan time 5 ms as shown in FIG. 11B, the decorrelation signal (intensity of the image) has linear relation with the blood flow of high speed, but signal from small vessels (slow flow) are lacking. In the image generated by data of inter-scan time 10 ms as shown in FIG. 11C, the decorrelation signal (intensity of the image) has linear relation with the blood flow of relatively low speed and signals of small vessels are fair, but decorrelation signal (intensity of the image) do not appear to have a linear relation with the blood flow of high speed, which is known as saturation. Combining data of both inter-scan time, a high (flow) dynamic range image can be abstained with possess both merits, fair representation of the small vessels and linear relation between decorrelation signals and blood flow for both slow and high flow rate as shown in FIG. 11D.

FIG. 11E shows another way to achieve multiple inter-scan time using different A-line (z-axis scan) period for B scans at different Y position. As shown in FIG. 11E, when the beam dwells at the j-th Y position, there are 2 repeated B scans executed by the fast (X) scanner. Assuming the A-line number per B scan is 512 and line period is 19.53 μs, the inter-scan time is 512×19.53 μs=10 ms. When the beam dwells at the (j+1)-th Y position, there are 2 repeated B scans executed by the fast (X) scanner. The A-line number per B scan is still 512 but the line period is 9.77 μs, the inter-scan time is 512×9.77 μs=5 ms. In the next Y position (J+2), the A-line period changes back to 19.53 μs. Therefore, alternating A-line period along Y positions can achieve 2 different inter-scan time. It is appreciable that alternating A-line period along Y positions will not introduce any motion artifact. For example, referring to FIG. 4B, from data acquired in each RBSSYP P=16 partial-spectrum APC variation images are generated, and the i-th image of these 16 images contributes to the frequency compounding at Y position of (j−1)*L+i, where i=1, 2, . . . . P and image number increases along the Y scanning direction. Therefore, at each Y position there are P divided by L (P/L)=8 images to be averaged for frequency compounding, including 4 images of inter-scan time=5 ms and 4 images inter-scan time=10 ms. In addition, frequency compounding can be done such that two 3D datasets are generated, wherein one 3D dataset is generated by averaging partial-spectrum frames of inter-scan time=5 ms and the other generated by averaging those of inter-scan time=10 ms. This can be simply achieved by averaging 4 partial frames of the same inter-scan time instead of 8 at each Y position.

FIG. 11F shows a model of decorrelation or reconstruction of angiograms after the DFT process, with high dynamic range, as a function of flow velocity with different inter-scan time. Dₘₐₓ is the mean saturated decorrelation signals, σ is the standard deviation of saturated decorrelation signals. DR_{ΔT} and DR_{ΔT/2} are the dynamic range of images acquired with inter-scan time of ΔT and ΔT/2. D_{ΔT} and D_{ΔT/2} refer to the decorrelation profiles of inter-scan time of ΔT and ΔT/2, respectively. α is the ratio between decorrelation signals acquired with ΔT (D_{ΔT}) and ΔT/2 (D_{ΔT/2}). It was assumed that over the decorrelation signal range of [σ, Dmax−σ], flow velocities and decorrelation signals are linearly related, where Dₘₐₓ is the mean saturation. Based on the assumption, the ratio between decorrelation signals acquired with ΔT (D_{ΔT}) and ΔT/2 (D_{ΔT/2}) is a constant, α. With the multiplication of the decorrelation profile of ΔT/2 with α, the new decorrelation profile, α*D_{ΔT/2}, has a higher saturation limit. The high dynamic range en face angiogram is reconstructed using both the angiogram acquired with ΔT and the angiogram corresponding to α*D_{ΔT/2}.

FIG. 11G shows histograms of image gray level acquired from FIGS. 11B, 11C, and 11D. Scale bars: 1 mm, evidencing the high dynamic range.

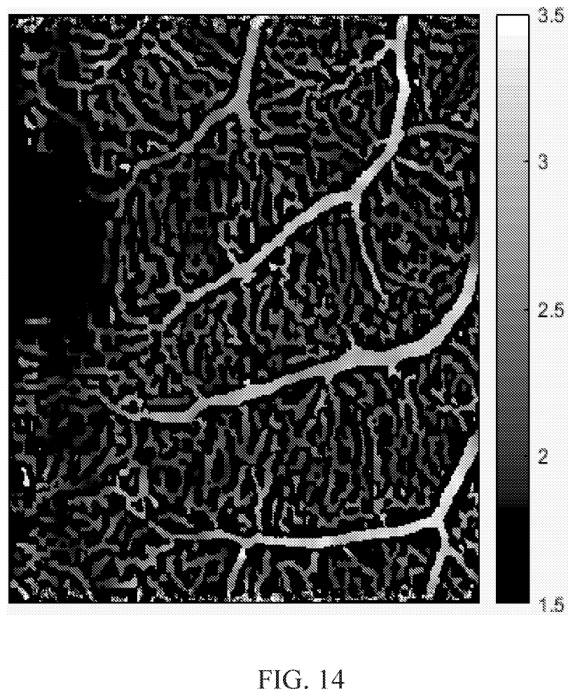

In some embodiments, a method to combine images of different inter-scan time may be in accordance with the steps described as follows, as further illustrated in FIG. 12:

1) Compute the mean standard deviation of decorrelation signal from blood vessels in the amplitude decorrelation image of inter-scan time T/2 (FIG. 11B) and that of inter-scan time T (FIG. 11C). The larger of two standard deviation is σ.

2) set a linear range to be maximum decorrelation −σ to σ, in which the decorrelation signal and flow speed have a linear relation for both amplitude decorrelation image of inter-scan time T/2 (T/2 decorrelation image, FIG. 11B) and that of inter-scan time T (T decorrelation image, FIG. 11C).

3) Generate a reference image for separating pixels into linear group and nonlinear group. This image can be T decorrelation image, the average of T/2 and T decorrelation image or the noise-reduced version.

4) The nonlinear group are those pixels in the reference image that is not in the range of maximum decorrelation −σ to σ. Remove all the pixels of nonlinear group in both T/2 decorrelation image and T decorrelation image. The new images is termed as linear T/2 decorrelation image and linear T decorrelation image, respectively.

5) Search for a mapping factor α, so that the histogram of α*linear T/2 decorrelation image is identical to the linear T decorrelation image. Alternatively, the histogram of linear T decorrelation image—α*linear T/2 decorrelation image is centered at zero.

6) To construct the high dynamic range image, for pixels that are in the linear group, the pixel intensity takes the corresponding pixel intensity of ½ (linear T decorrelation image+α*linear T/2 decorrelation image); for pixels in the reference image with intensity below σ, the pixel intensity takes the corresponding pixel intensity of the original T decorrelation image; for pixels with intensity above maximum decorrelation—σ, the pixel intensity takes the corresponding pixel intensity of α*original T/2 decorrelation image.

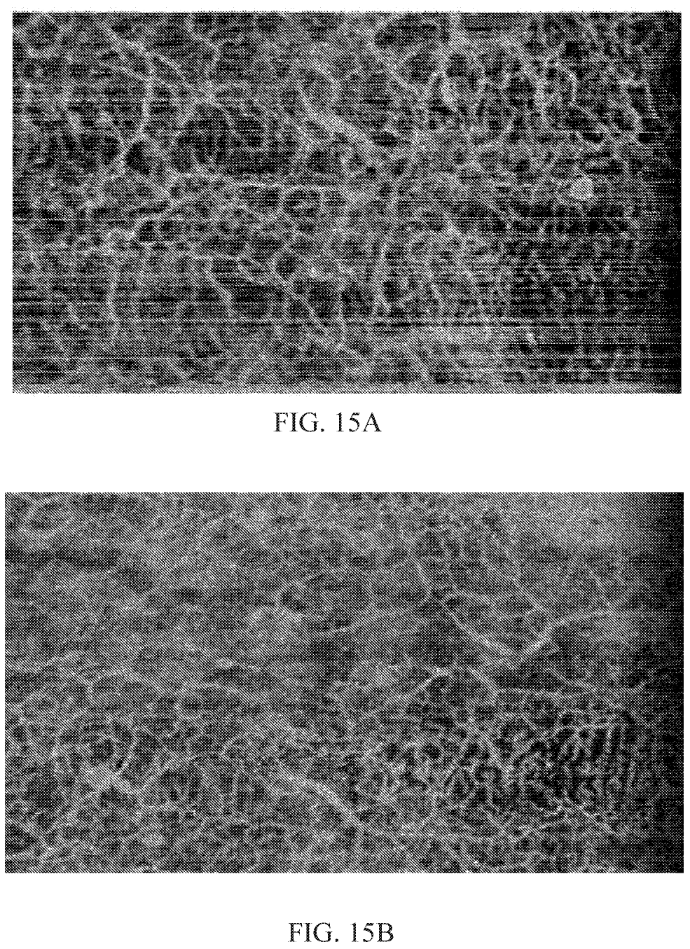

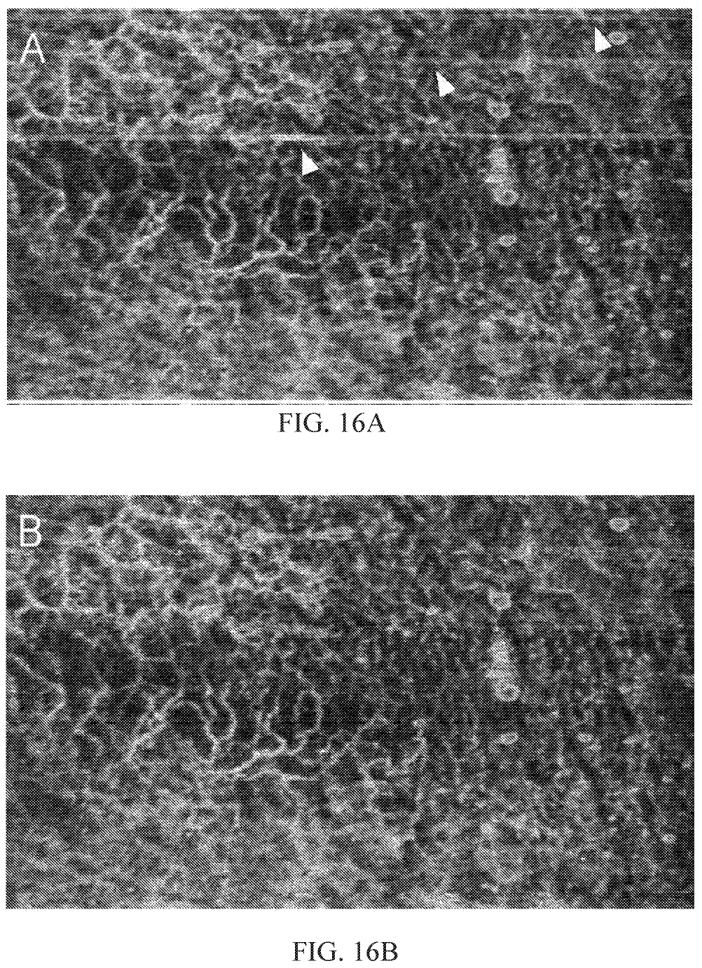

With the partial B-frame scanning protocol mentioned above, four inter-scan time may be realized with two B-scan repeats. In an example, blood flow over a small FOV of 1.7 mm×1.7 mm is imaged in the human eye in vivo as shown in FIG. 13. The gray level represents relative blood flow velocity. The acquisition time was 4 second. Using the dynamic range improvement method mentioned above and the amplitude decorrelation of inter-scan time T and T/2 as shown in FIG. 12, the images of four inter-scan time in FIG. 13 are merged into a high dynamic range image in which intensity is approximately linearly proportional to the blood flow velocity over a range of around 0 to 2 mm per second, as shown in FIG. 14.

It is contemplated that the system 200A may be applied to various applications, such as, but not limited to, OCTA.

The OCT or imaging system 200A or 200B may be configured for various light sources of different wavelength. In some embodiments, the system 200B employs a near-infrared light source with centre wavelength of 1020 nanometers (nm) and a full spectral bandwidth of 166 nm. The A-line (z-axis or depth axis scan) rate was set at 30,720 Hz with 1024 A-lines per B-scan. The focal length of the collimation lens (L1) 222 is 15 mm and that of the objective lens (L2) 224 in the sample arm is set at 75 mm. At each slow scanning (y) location, N=2 fast axis (x) scans were implemented, i.e. each B-scans contains data generated from 2 fast axis (x) scans.

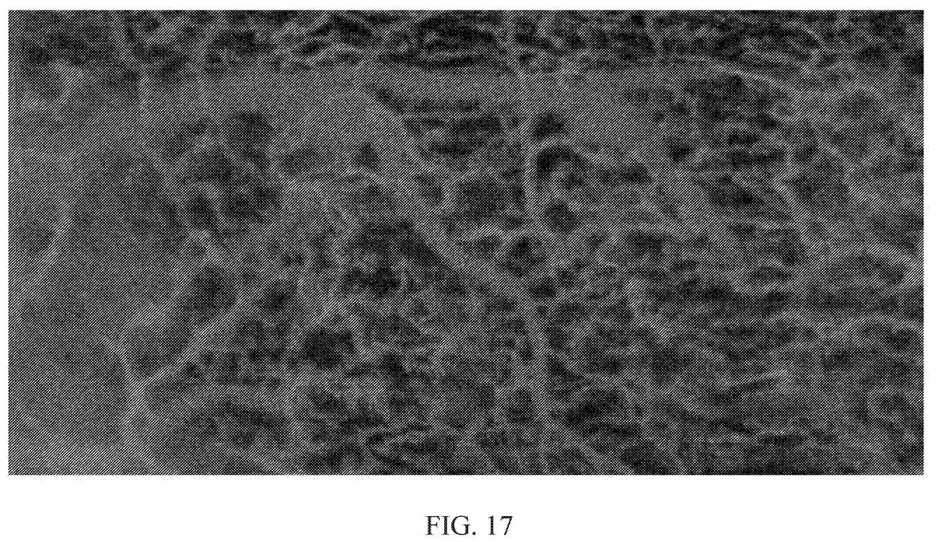

In a study involving the system 200B, and with reference to FIG. 15A, where the number of spectral bands denoted K is equal to the number of spectral bands in inter-scan distance L, using the dispersive element 230 in the form of an equilateral prism (material: N-SF11), the collimated sample beam is dispersed into a fan with fan angle of 0.0126 radian, and the extended source at the focal plane of the objective lens has a length E=0.945 mm. The n1-n2 plane is rotated around the BC principle ray so that the angle between the extended source on y scanning mirror and the rotational axis of y scanner is θ=2.0°. The y projection of the extended source E_{Y}=E*sin θ=0.033 mm. The inter-scan distance is set to be E_{Y} and L=5. The full spectrum is split in to K=L=5 equally-spaced spectral bands, and each spectral band is further split into M=6 sub-spectral bands using Hanning windows. For each of 5 spectral bands, the data processing is the same as SSADA with N=2 and M=6.

The preliminary result without time averaging is presented in FIG. 15A. The number of B-scans were 200, which generate (200/N)*L=500 OCTA frames. For the standard SSADA, to generate 500 OCTA frames with the same B-frame size, the number of B-scans has to be 500*N=1000. That is to say the OCT system 200A, 200B is 5 times faster or the field of view (FOV) of the OCT system 200A, 200B is 5 times larger than the existing technology, with all other imaging performance the same. Since K=L, it is appreciable there is no overlap between two adjacent RBSSYP and temporal averaging. The connectivity of the blood vessel network is not optimal due to spectral variations between adjacent OCTA frames. In addition, there are motion artifacts manifested as horizontal lines.

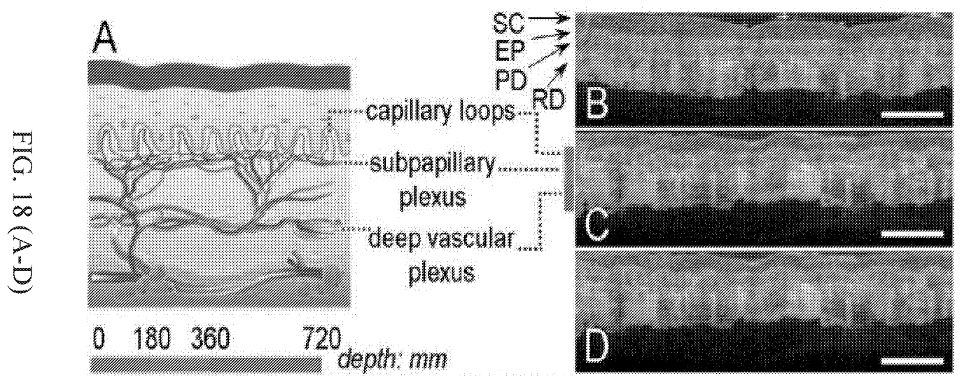

In another study using system 200B and with reference to FIG. 15B, FIGS. 16A and 16B, the value of L and K are different, where L=2, K=10. Using the dispersive element 230 in the form of an equilateral prism (material: N-SF11), the collimated sample beam is dispersed into a fan with fan angle of 0.0126 radian, and the extended source at the focal plane of the objective lens has a length E=0.945 mm. The n1-n2 plane as illustrated in FIG. 5 is rotated around the BC principle ray so that the angle between the extended source on y scanning mirror and the rotational axis of y scanner is θ=3.7°. The y projection of the extended source E_{Y}=E*sin θ=0.061 mm. The inter-scan distant is set to be E_{Y}/2, L=2 and K=10. The full spectrum is split in to k=10 equally-spaced spectral bands, and each spectral band is further split into M=2 sub-spectral bands using Hanning windows (function). For each of 5 spectral bands, the data processing is the same as SSADA with N=2 and M=3. Temporal averaging is done with partial-spectrum OCTA frames obtained from 4 consecutive RBSSYP. For example, the partial-spectrum OCTA frame of the 7th spectral band of the 1st RBSSYP, the partial spectrum OCTA frame of the 5th spectral band of the 2nd RBSSYP, the partial-spectrum OCTA frame of the 3rd spectral band of the 3rd RBSSYP, and the partial-spectrum OCTA frame of the 1st spectral band of the 4th RBSSYP are averaged to generate the final OCTA frame at the y index of 7.

The preliminary result for (K=10, L=2) is presented in FIG. 15B. Compared with a prior art SSADA system having the same exposure time, B-frame size, and scanning duration, the FOV of FIG. 15B is found to improve by a factor of 2. Since temporal averaging is implemented. The motion artifacts are largely suppressed. Temporal averaging does not remove all the motion artifacts. As shown in FIG. 16A, some motion artifacts (arrow heads) caused by a large motion can be further removed by rejecting low quality partial-spectrum OCTA frames before temporal averaging (FIG. 16B).

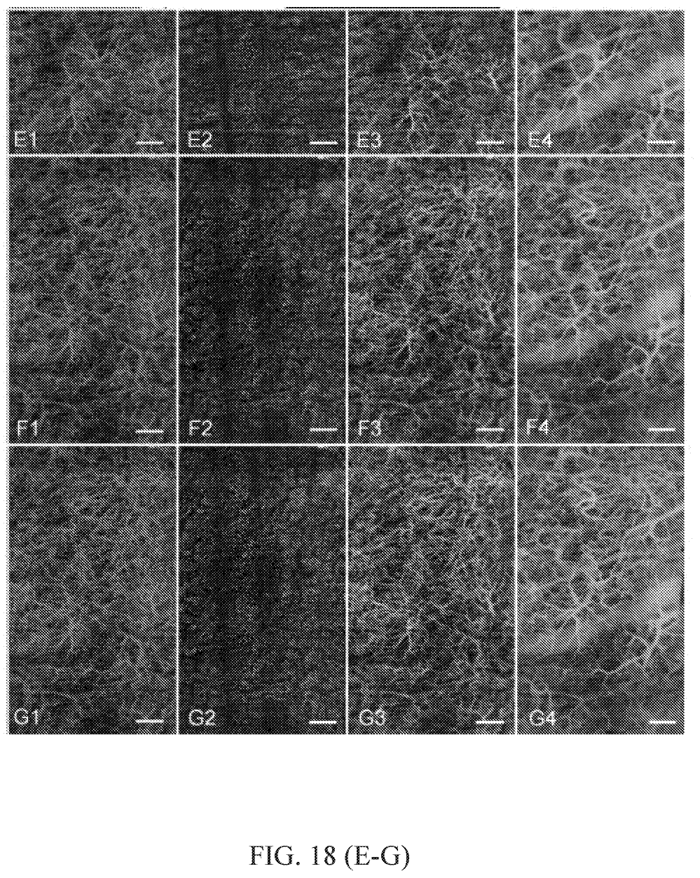

In another study using system 200B and with reference to FIG. 17, the value of L and K are different, where L=1, K=62. Using the dispersive element 230 in the form of an equilateral prism (material: N-BK7), the collimated sample beam is dispersed into a fan with fan angle of 0.0126 radian, and the extended source at the focal plane of the objective lens has a length E=0.42 mm. The n1-n2 plane of FIG. 5 is rotated around the BC principle ray so that the angle between the extended source on y scanning mirror and the rotational axis of y scanner is θ=90°. The y projection of the extended source E_{Y}=E*sin θ=0.42 mm. The inter-scan distant was set to be E_{Y}/62, L=1 and K=62. The full spectrum is split in to K=62 equally-spaced spectral bands, and each spectral band is further split into M=15 sub-spectral bands using Hanning windows. For each of 62 spectral bands, the data processing is the same as SSADA with N=2 and M=15. Temporal averaging is done with partial-spectrum OCTA frames obtained from 62 consecutive RBSSYP. As shown in FIG. 17, the final OCTA is free from any motion artifacts.

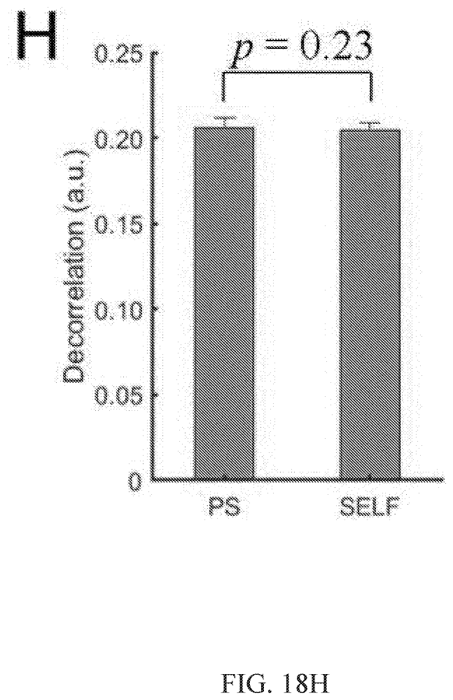

In another study and with reference to FIG. 18, a comparison of field of view (FOV) between a prior art method of point scanning and the extended source scanning of the OCT system 200A, 200B with the same A-line rate and total acquisition time were illustrated. FIG. 18A shows the schematic of a sample human skin structure of a skin vasculatures at the palm side of the proximal interphalangeal (PIP) joint of the middle finger of healthy human subjects and vasculature under study. FIG. 18B shows a representative cross-sectional OCT structural image acquired using the point-scanning scheme overlaid with blood flow signals, and where SC: stratum corneum; EP, epidermis; PD, papillary dermis; RD, reticular dermis. FIG. 18C & FIG. 18D shows the corresponding images acquired using spectrally extended line field scheme before (FIG. 18C) and after (FIG. 18D) Y-deconvolution. The three bars (from 0 to 720 mm) correspond to the depth range of three en face slabs. FIG. 18E (comprising E1-E4) show en face OCTA images acquired with the point-scanning scheme in the proximal interphalangeal joint of the middle finger (palm side) in vivo: E1, en face projection of three slabs color coded by depth, E2, the first slab mainly containing capillary loops, E3, the second slab mainly containing subpapillary plexus, E4, the third slab containing deep vascular plexus. FIG. 18F comprising F1-F4 show en face images obtained by the extended mode scanning of the system 200A, 200B, acquired at the same skin area corresponding to E1-E4 before Y deconvolution. FIG. 18G comprising G1-G4 show en face images obtained by the extended mode scanning of the system 200A, 200B, corresponding to F1-4 after Y-deconvolution. (H). No statistically significant difference in decorrelation between the point-scanning (PS) and OCTA images generated by the system 200A, 200B were observed. Scale bars: 1 mm.

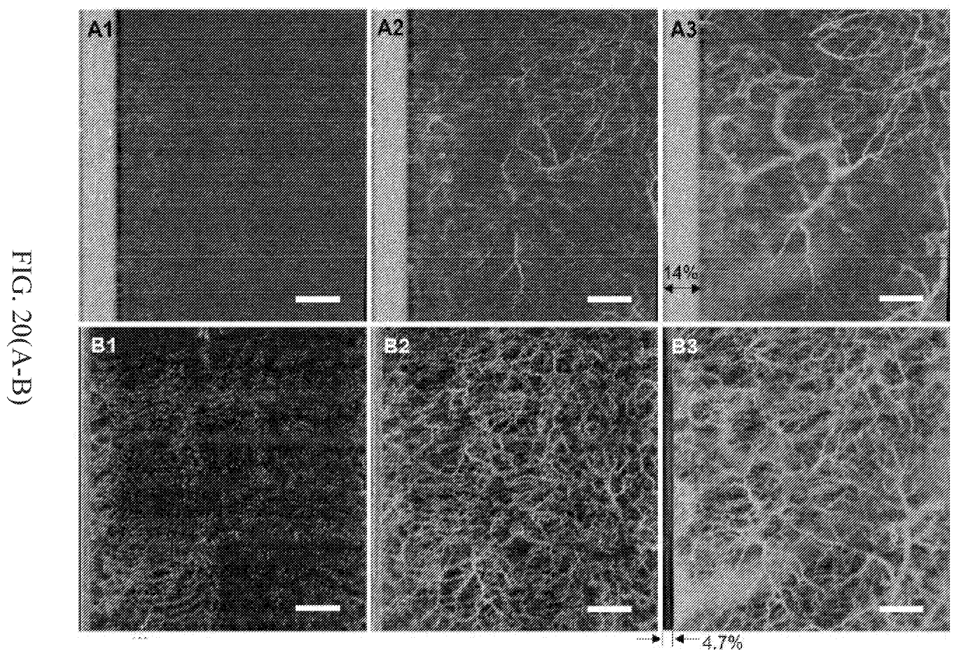

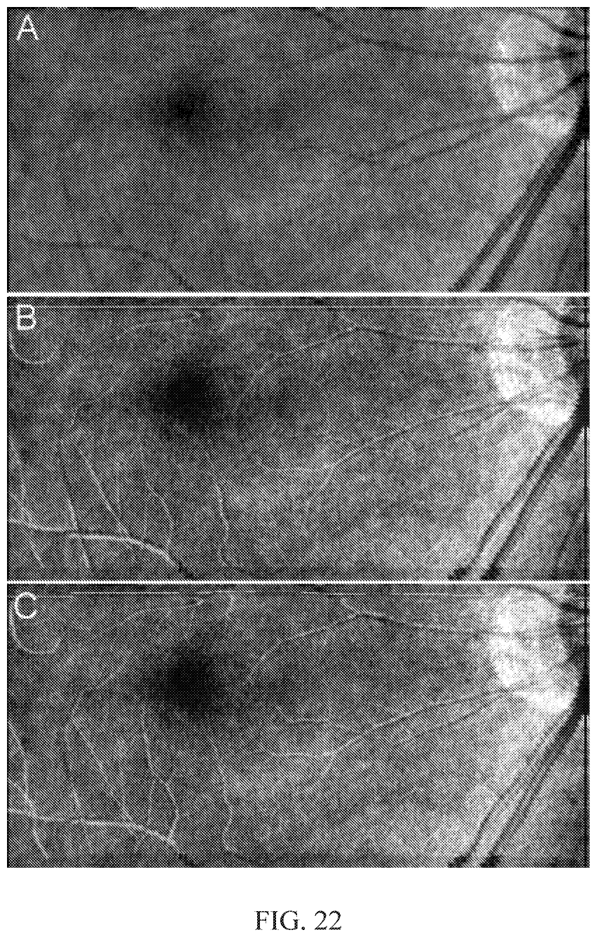

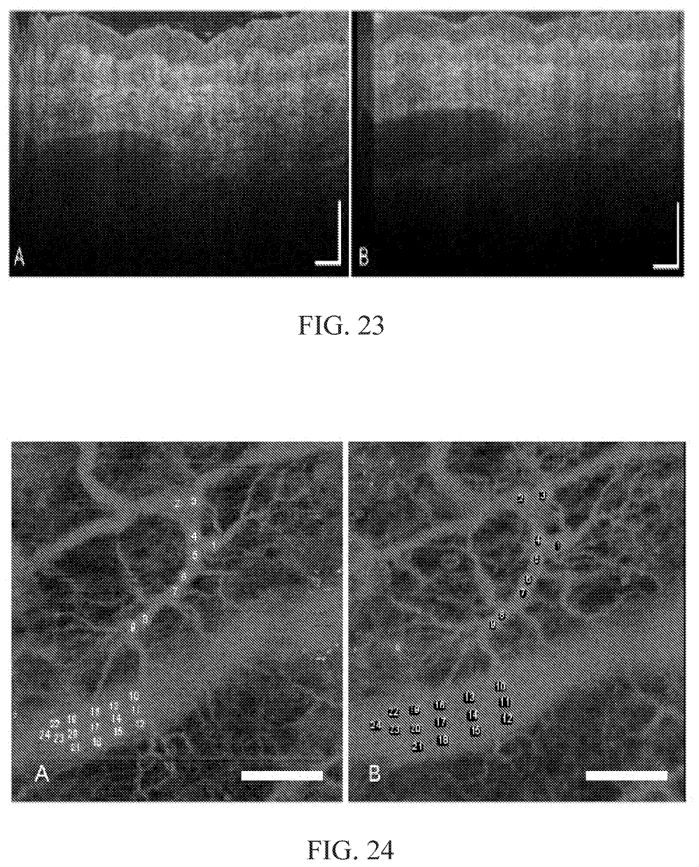

In FIG. 18, the FOV between two schemes (prior art point scanning and the extended source scanning of the OCT system 200a, 200b μsing sample arm 220, i.e. the “extended source scheme”) under the same conditions: 512 A-lines per B-frame, 400 Y-scan positions, and a total acquisition time of 4.096 seconds are compared (see also FIG. 10). The FOV scanned with the point scanning scheme is 6.55 mm×5.12 mm (FIGS. 18E1-E4). The images obtained by the extended scanning scheme of the system 200A, 200B provides twice as large FOV when the inter-scan distance is doubled as illustrated in FIG. 2B (See FIG. 18F1-18F4& FIG. 18G1-18G4). With the same display contrast, the image quality of the extended source scheme is comparable to that of the point-scanning scheme. First, through one-to-one comparison the images of vascular microstructures are almost identical between the point-scanning and extended source scanning using sample arm 220, including the capillary loops (FIGS. 18E2, 18F2 & 18G2). The en face images generated by the extended source scanning using sample arm 220 are slightly less crispy before deconvolution because of the convolution effect mentioned in FIGS. 18F1-18F4). Nevertheless, this relatively insignificant issue is corrected after deconvolution as shown in FIGS. 18G1-18G4). Secondly, the penetration depth is also comparable as can be seen in the en face images of deep vascular plexus (FIGS. 18E4, 18F4 & 18G4), and cross-sectional angiograms (FIGS. 18B-18D, and FIG. 23). Thirdly, the mean decorrelation, measured from one-to-one matched vascular areas (FIG. 24), are also comparable between the point-scanning scheme (0.206±0.006) and the extended source scheme (0.204±0.004), as shown in FIG. 18H) (student's t-test, p=0.23). The speckle contrast of en face images generated by the extended source scanning of the OCT system 200a, 200b μsing sample arm 220 (0.439±0.059) is close to that of the point scanning scheme (0.353±0.036), although the number of partial-spectrum decorrelation frames to be averaged at each Y image position is half of that of the point-scanning scheme. This relatively low speckle contrast was attributed to the fact that the partial-spectrum decorrelation frames at a Y image position are acquired at different time. Comparing the images, which is also applicable for FIG. 20, while there are visible motion artifacts seen in the en faces images associated with the point-scanning scheme, the corresponding images obtained from the system 200A, 200B of the present disclosure are shown to be almost free from such artifacts, because motion induced signal deviations are distributed into 16 Y image positions, substantially damping the contrast of motion artifacts. This artifact damping mechanism is analogous to a selective low-pass filter along Y direction, which does not affect the signal.

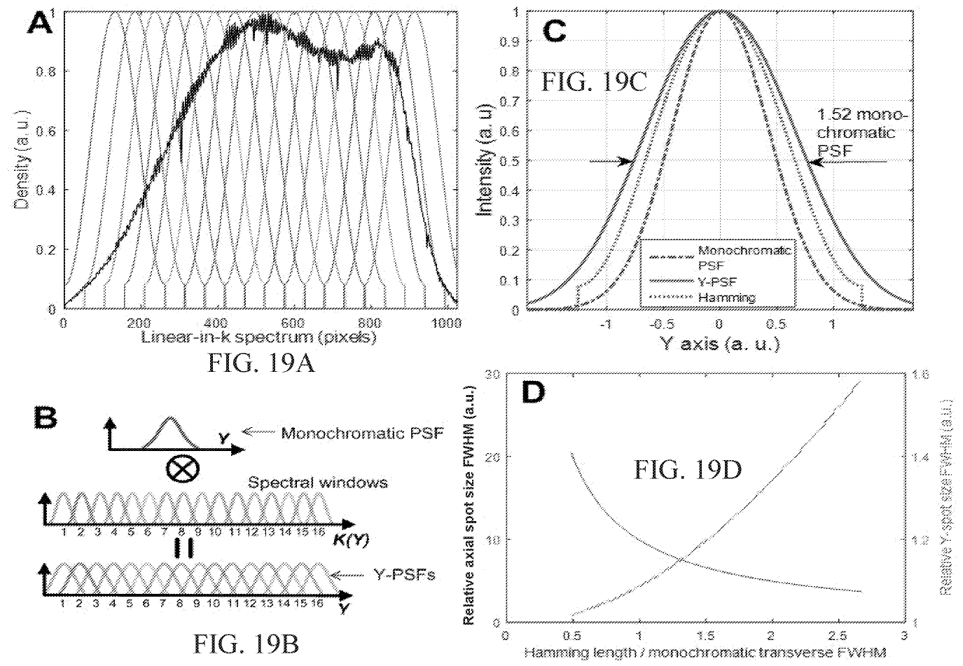

In another study and with reference to FIG. 19A to 19D, sixteen Hamming windows are configured with size of 263 pixels and spacing of 52 pixels to generate 16 spectral bands in k-space respectively, as shown in FIG. 19A. The axial resolution (FWHM) of each band was measured to be about 28.5 μm in tissue, with a refractive index of 1.38 as shown in FIG. 19C. The transverse resolution (FWHM) of each band was measured to be 39.6 μm (10-90% width of an edge scan) because of the convolution between the Hamming window and monochromatic point-spread function (PSF) as shown in FIG. 19B, which is 1.51 times larger than the monochromatic transverse resolution and agree well with theoretical prediction (FIG. 19C). The trade-off between axial and transverse resolution along Y-axis is characterized in FIG. 19D.

The theoretical transverse spot size at 1310 nm is about 27 μm (full-width at half maximum, FWHM) since the nominal mode field diameter of SMF-28e fibre is 9.2 μm at 1310 nm. The monochromatic transverse resolution at 1310 nm was approximated to be 26.2 μm by measuring 10-90% width of an edge scan using the part of signal at the centre of the spectrum with a narrow line width (about 1.5 nm FWHM), see FIG. 19D. For a Gaussian spot, the lateral resolution, defined at its e⁻² radius, can be shown to be 0.78 times the 10-90% edge width, so that the monochromatic spot size (FWHM) is estimated to be 24.1 μm.

For deconvolution along Y direction, an iterative procedure for recovering an underlying image that has been blurred by the point-spread function, such as the Lucy-Richardson method with a deconvlucy function in MATLAB®, was used. A Hamming window mentioned above as the point-spread function (PSF in deconvlucy) and damping threshold of 2.

In another study and with reference to FIGS. 20A to 20E, there is a comparison of vessel visibility and noise level between a prior art point scanning and the OCT system 200A. 200B with the same input power and total acquisition time. FIG. 20A1-A3 shows an en face OCTA projection of skin slab at the depth of capillary loop (FIG. 20A1), subpapillary plexus (FIG. 20A2) and deep vascular plexus (FIG. 20A3) acquired with the prior art point-scanning scheme running at 80,384 Hz A-line rate. FIG. 20B1-20B3 show the corresponding OCTA images with L=4 acquired at 22,000 Hz A-line rate based on the OCT system 200A, 200B. FIG. 20C shows a signal to noise ratio (SNR) as a function of exposure time. SNRel, SNRRIN, and SNRshot indicate ratio of signal to electrical noise, relative intensity noise, and shot noise, respectively. It is to be appreciated that SNRel=SNRrin for optimal total SNR. Dot, square and diamond indicate measured SNR values, respectively. FIGS. 20D and 20E show magnified views of a region of interest in A2 and B2 with inter-scan time at 6.4 ms and 23 ms, with scale bars: 1 mm. The results indicate relatively clearer images of the respective vessels obtained based on the OCT system 200A, 200B.

It is appreciable that there are always visible motion artifacts, which may appear as thin bright and dark lines, in en face angiograms acquired with the point-scanning scheme (FIGS. 18E1-18E4, FIGS. 20A1-20A3). In contrast, the corresponding images captured by the extended source scheme are almost free of such motion artifacts, because the motion induced signal deviations are distributed into a plurality of, exemplified by sixteen (16) Y image positions, substantially damping the contrast of motion artifacts (FIGS. 18F1-18F4 & 18G1-18G4, FIG. 20B1-20B3). The artifact damping mechanism is analogous to a selective low-pass filter along Y direction, which does not affect the signal. In a separate experiment, motion artifacts were deliberately generated by removing a hand rest/support holding a sample hand, before image acquisition. Corresponding motion artifacts generated in extended source scanning using sample arm 220 of the OCT system 200a, 200b, en face images appear as low-intensity variations in the background. The extended source scheme allows tailoring exposure time and inter-scan time without affecting FOV or total acquisition time. To validate the performance of the extended source scheme, a 3D dataset was acquired with the point-scanning scheme with a nominal FOV of 6.55 mm×6.55 mm and a total acquisition time of 3.28 s. The inter-scan time was 6.4 milliseconds (ms) with an A-line rate of 80,384 Hz and 512 A-line per B-frame (FIGS. 20A1-20A3). In the extended source scheme, the inter-scan distance to be 4Δx and the A-line rate to be 22,000 Hz, so that a 3.65 times longer inter-scan time and the same nominal FOV within 2.98 s were achieved (FIGS. 20B1-20B3). The advantage of longer inter-scan time is the significantly increased sensitivity to slow flow in small vessels and capillaries, which are largely invisible in the point-scanning OCTA images due to relatively shorter inter-scan time as shown in FIGS. 20A1-20A3, 20B1-20B3, 20D & 20E. Notably, a practical advantage of longer integration time is about 10% larger X-scan duty cycle (FIGS. 20A3 & 20B3). In addition, most SD-OCT devices are RIN and electrical noise limited at working A-line rate. In the current device, total SNR is measured to be 9.94 dB lower at 80,384 Hz than that at 22,000 Hz A-line rate (FIG. 20C), which can be approximately broken down to 5.84-dB drop in signal due to reduced exposure time and 4.1-dB drop in signal to RIN ratio (SNRRIN). This SNRRIN drop significantly elevates the noise background and overwhelms weak vessel signals from small vessels (FIGS. 20A1-20A3 & FIG. 20D) compared with images captured using the extended source scheme (FIGS. 20B1-20B3 & FIG. 20E).

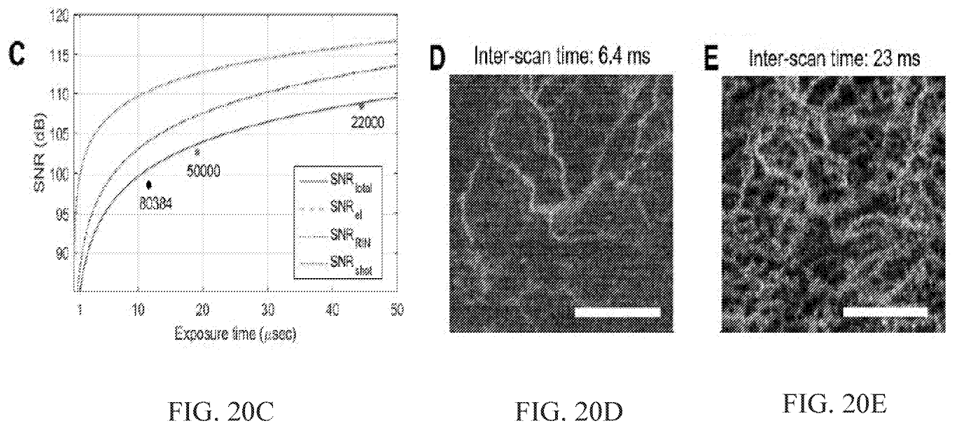

In another study and with reference to FIGS. 21A to 21D, representative images acquired using the standard OCT and the OCT system 200A, 200B within the same acquisition time, respectively. The image size shown in FIG. 21A is 6.5 mm×3.8 mm, and that shown in FIG. 21B is 6.5 mm×7.6 mm. In FIGS. 21C and 21D, a representative OCTA image is shown before and after motion correction respectively. Image size: 6.5 mm×6.5 mm. The comparison indicate a twice as large FOV captured for the OCT system 200A, 200B within the same acquisition time due to the inter-scan time of N=2, and an image after motion correction.

In the study shown in FIGS. 21A to 21D, the capability of the extended source scheme is further validated using a spectral domain OCT setup that supports switching between the standard point scanning mode and the extended source scanning mode. The A-line rate was set to be 50 k Hz and B-scan repetition number N=2 for all the experiments. A 2 times faster imaging speed was demonstrated with extended source scanning on human skin in vivo (FIGS. 21A & 21B). By comparing the mean amplitude decorrelation from the same blood vessels, extended source scanning is shown to be comparable to the standard point scanning in signal strength (Data not shown). There is a trade-off between the Y-transverse resolution and axial resolution, which is similar to the spectral-spatial resolution trade-off in short time Fourier transform. With Y-transverse resolution degraded by 1.1565, the axial resolution is degraded by about seven times. Since 8 partial-spectral decorrelation frames are generated in different Y scan cycles, the extended source scanning supports variable inter-scan time (3.75 ms and 7.5 ms) with N=2 (Data not shown). Making use of the overlap region between illumination areas of 2 adjacent B-scans at distinct Y positions, it is additionally demonstrated that the extended source scanning is capable of correcting slow sample motion using OCTA data without aid of hardware tracking FIGS. 21C & 21D). Note that in FIGS. 21C & 21D, the white line marked ‘W’ is shadow of a straight metal wire, which serves as the reference for motion correction and excluded in the motion calculation.

It is contemplated that the higher imaging speed is enabled by the extended source scanning may mitigate the field of view problem in ophthalmic OCTA. The capability of providing variable inter-scan time with N=2 will resolve the trade-off between imaging speed and flow dynamic range. The motion correction function of extended source scanning may help to reduce imaging time and remove motion artefacts in the slow axis. It is contemplated that optimized solutions may be developed to mitigate axial and transverse resolution degradation according to clinical needs.

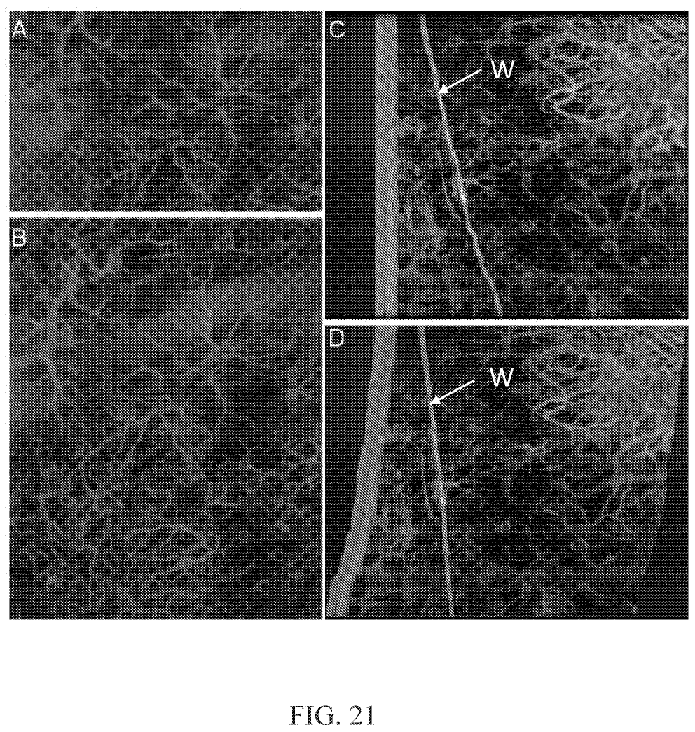

In another study and with reference to FIGS. 22A to 22C, en face projection of OCT structural images were acquired within a time of 1.18 seconds. FIG. 22A shows the OCT in vivo structural image of a retina, FIG. 22B shows the en face OCTA image before motion correction merged with OCT structural image, and FIG. 22C shows the en face OCTA image after motion correction merged with OCT structural image. The en face angiogram after motion tracking and correction was found to match better with the en face projection of OCT structural data than the uncorrected, hence validating the effectiveness of the motion artifact correction method as described.

In a study related to FIG. 18 and with reference to FIGS. 23A and 23B, cross-sectional structural images in the form of amplitude frames of a skin vasculatures at the palm side of the proximal interphalangeal (PIP) joint of the middle finger of healthy human subjects and vasculature obtained using a prior art point scanning OCT (see FIG. 23A) and the extended source scanning of the OCT system 200A, 200B (see FIG. 23B) are shown, with scale bars: 0.5 mm, with the axial resolution of the OCT system 200A, 200B cross-sectional images comparable to that of the point-scanning.

In another study and with reference to FIGS. 24A and 24B, regions of interest for comparison of decorrelation between a prior art point-scanning (FIG. 24A) and the extended source scanning based on OCT system 200A, 200B (FIG. 24B) were indicated. The mean decorrelation, measured from one-to-one matched vascular areas are found to be comparable between the point-scanning OCTA (0.206±0.006) and OCTA images obtained based on the system 200A, 200B (0.204±0.004) and with reference to FIG. 18H.

It is appreciable that the results obtained in the various aforementioned studies may be based on the OCT system 200A, 200B configured with specific components as follows: two superluminescent diode modules (IPSDS1313 and IPSDS1201C, Inphenix, CA, USA) with a 50:50 fibre coupler (TW1300R5A2, Thorlabs Inc, USA). The combined light source 202 is configured to provide a radiation having a wavelength in a range from 1230 nm to 1360 nm (−6 dB). The output of the fibre coupler are connected to two optical circulators (PIBCIR-1214-12-L-10-FA, FOPTO, Shenzhen, China), which guide the light beams to the sample arm 220 and reference arm 250, respectively. The light back reflected from the reference arm and back-scattered from the sample arm are combined using a 95:5 fibre coupler (WP3105202A120511, AC Photonics, CA, USA).

The prior art point scanning scheme may be configured using a conventional point scanning scheme used as a basis for comparison with the system 200A, 200B of the present disclosure. In the point scanning scheme, the sample beam is firstly collimated by an achromatic lens L1 (AC050-010-C. Thorlabs Inc., USA) before reflected by a mirror (RM) and a pair of galvanometer scanners and focused by an objective lens L2 (AC254-050-C, Thorlabs Inc. USA). To test the effectiveness of the system 200B relative to a conventional point scanning setup/scheme, the mirror (RM) is replaced by a set of three identical prisms (N-SF11, PS872-C, Thorlabs Inc.) 230 with apex angle of 30° and angular spacing of about or substantially 56.9°. Switching between the point scanning scheme and scanning scheme of using the sample arm configuration 220 may be realized with the manual translation stage 232.

The three identical prisms 230 may be coated with anti-reflection material so that one-way transmission efficiency of three prisms was measured to be 94%. The polychromatic sample beam is dispersed by the prisms 230 into a line along the slow (Y) axis in the focal plane of the objective lens (L2) 224. The spectrometer 270 may be comprised of a collimating lens L5 (AC254-035-C. Thorlabs Inc., USA), the transmission grating (PINGsample-106, Ibsen Photonics, Denmark) 272, a home-made multi-element camera lens (not shown) and a line scan camera (LDH2, Sensors Unlimited, USA) 276. The camera pixel size is 25 μm by 500 μm (width by height). All 1024 pixels may be utilized, and the total spectrometer efficiency was measured to be 0.61, including the quantum efficiency of the camera. The spectral resolution is 0.148 nm, resulting in a total ranging depth of 2.89 mm in air. The axial resolution was measured to be 9.82 μm in air. The 6-dB ranging depth was measured to be 1.6 mm in air and the sensitivity roll-off over depth is ˜3.75 dB/mm. With the optical power incident on the sample being 4.74 mW, the sensitivity measured at ˜150 μm from DC is 108.52 dB, 102.56 dB, and 98.58 dB at the A-line rate of 22 k Hz, 50 k Hz, and 80 k Hz, respectively, this was found to agree with the theoretical predictions.

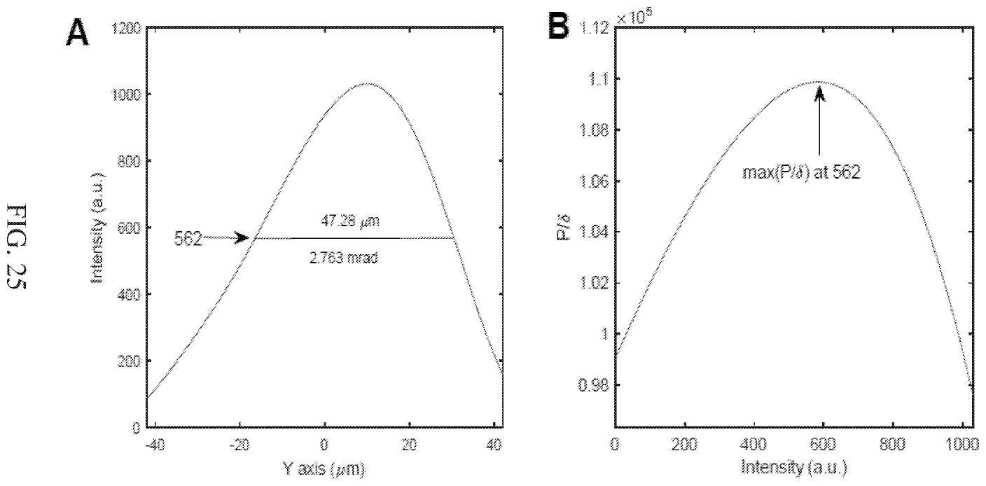

FIG. 25A show an intensity profile of the line field that was estimated using the light source spectrum captured by the spectrometer 270 of focus along a Y-axis. FIG. 25B shows a ratio between partial power (P) within window width (δ) based on a “Most Restrictive Ratio” method. The window width is 159 μm (3.176 mrad) when the ratio reaches the maximum. The transverse positions of spectral bands were calibrated using a USAF 1951 resolution chart.

The extended source correction factor CE is (2.763±1.5 mrad)/2/1.5 mrad=1.421. The power limit calculated using CE applies to the partial power within the angular subtense 8, instead of the total power. There are 27.02% of total power that is outside the angular subtense 8. For the experiments conducted with the point-scanning scheme and total image acquisition time of 4.7 s, the optical power incident on a cornea used for a study is 1.754 mW. The corresponding power for the scanning of the present disclosure is calculated as (1.754 mW×CE)/(1−0.2702)=3.415 mW. A power of about 3.20 mW may be used for the one or more of the aforementioned studies.

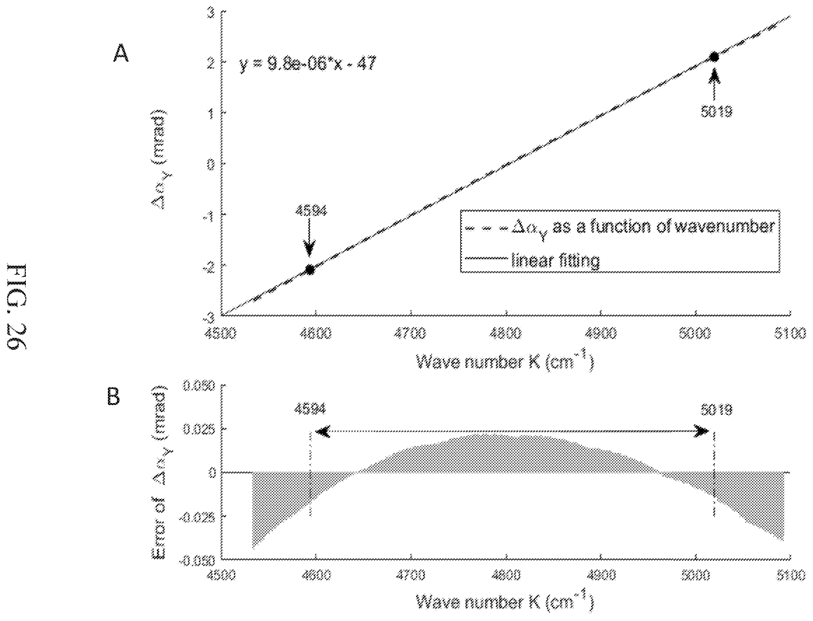

FIG. 26A shows a plot of an angular substance along Y-axis (ΔαY) as a function of wave number (K) (dash line). The solid line is the linear fitting. The centre wave number of the 1st and 16th spectral band are 4594 cm⁻¹ and 5019 cm⁻¹, respectively. FIG. 26B shows a linear error of ΔαY is <0.0217 mrad within the range from 4594 cm⁻¹ to 5019 cm⁻¹, corresponding to <1.0855 μm in the focal plane of the objective lens.

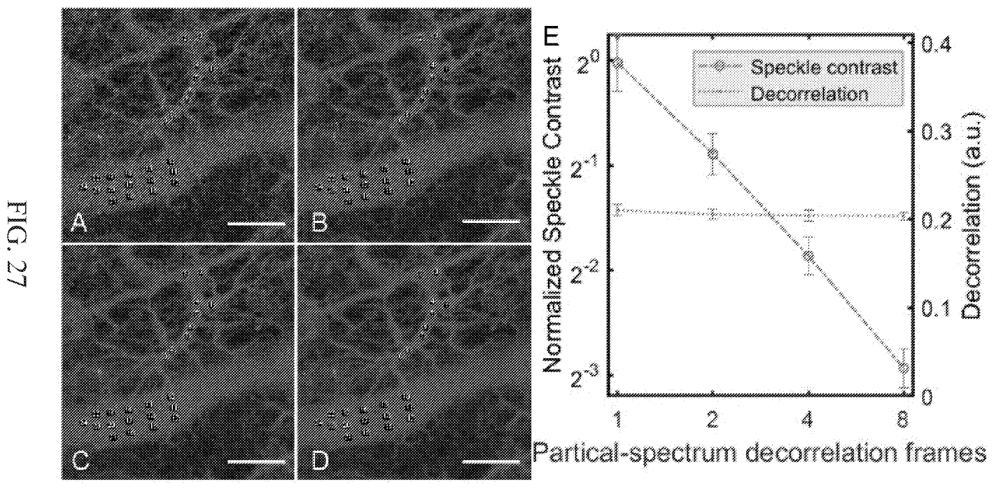

FIGS. 27A to 27D show regions of interests (ROIs) on OCTA images obtained from the dependent on the number of partial spectrum decorrelation frames at each Y image position, and FIG. 27E is a plot of a speckle contrast (normalized) and decorrelation against the number of partial spectrum decorrelation frames. FIG. 27A shows the ROIs for speckle contrast with averaging 1 partial-spectrum decorrelation frame (3rd frame); FIG. 27B shows the ROIs for speckle contrast with averaging 2 partial-spectrum decorrelation frames (3rd and 7th frames); FIG. 27C shows the ROIs for speckle contrast with averaging 4 partial-spectrum decorrelation frames (1st, 3rd, 5th, and 7th frames); FIG. 27D shows the ROIs for speckle contrast with averaging 8 partial-spectrum decorrelation frames (all 1st to 8th frames). The plot shown in FIG. 27E indicates that the speckle contrast of the OCTA signals obtained based on system 200A, 200B is inversely proportional to the number of partial-spectrum decorrelation frames in frequency-temporal compounding. Scale bar: 1 mm.

In the FIGS. 27A to 27D, it is appreciable that deconvolution works well in restoring lateral resolution in Y-axis. As deconvolution is an intensity-based model, there have been concerns when phase information is involved in the 3D coherent image formation. Other concerns of using deconvolution in imaging scattering tissues are that the optical transfer function may not be accurate and that it is sensitive to speckle and noise. In the extended source scanning mode of system 200A, 200B, artifacts associated with deconvolution are unnoticeable due to the following: First, the convolution involves phase information only in the depth dimension. Second, the speckle contrast is substantially reduced by frequency-temporal compounding as speckles of partial spectrum decorrelation frames are fully uncorrelated. Thirdly, the optical transfer function is accurate since convolution is done digitally.

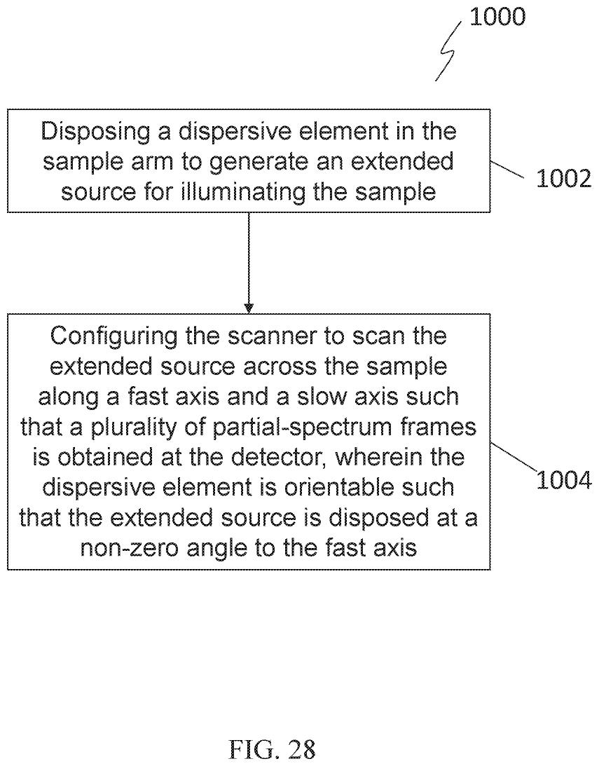

It is contemplated that the prior art system 100 may be modified to achieve the present disclosure based on the following method 1000 for modifying an optical coherence tomography (OCT) system or device, with reference to FIG. 28, comprising the steps of: disposing a dispersive element in the sample arm to generate an extended source for illuminating the sample (1002); configuring the scanner to scan the extended source across the sample along a fast axis and a slow axis such that a plurality of partial-spectrum frames is obtained at the detector (1004); wherein the dispersive element is orientable such that the extended source is disposed at a non-zero angle to the fast axis.

While the disclosure has been particularly shown and described with reference to specific embodiments, it should be understood by those skilled in the art that various changes in form and detail may be made therein without departing from the spirit and scope of the disclosure as defined by the appended claims. The scope of the disclosure is thus indicated by the appended claims and all changes which come within the meaning and range of equivalency of the claims are therefore intended to be embraced.

1. An optical coherence tomography (OCT) system for imaging of a sample, comprising:
a sample arm for directing light onto the sample, the sample arm comprises sample arm optics comprising a dispersive element to generate an extended source for illuminating the sample;
a reference arm;
a detector for detecting an interference signal from light that is reflected from the reference arm and light that is back-reflected or back-scattered from the sample; and
a scanner for scanning the extended source across the sample along a fast axis and a slow axis such that a plurality of partial-spectrum frames is obtained at the detector;
wherein the dispersive element is orientable such that the extended source is disposed at a non-zero angle to the fast axis.

2. The system according to claim 1, wherein the non-zero angle is an acute angle.

3. The system according to claim 1, wherein the non-zero angle is a right angle.

4. The system according to claim 1, wherein the scanner is configured to scan a plurality of times along the fast axis at each of a respective plurality of positions along the slow axis.

5. The system according to claim 1, comprising at least one processor configured to generate at least one OCT image and/or at least one optical coherence tomography angiography (OCTA) image from the partial-spectrum frames.

6. The system of claim 5, wherein the at least one processor is configured to check for at least one low quality frame of the partial-spectrum frames and remove the at least one low quality frame prior to generating the at least one OCT image and/or the at least one OCTA image.

7. The system claim 5, wherein the scanner is configured to scan a plurality of times along the fast axis at each of a respective plurality of positions along the slow axis and wherein the at least one processor is configured to perform temporal averaging of the partial-spectrum frames for each of the respective plurality of positions to generate a plurality of respective OCT images associated with the respective plurality of positions.

8. The system according to claim 7, wherein the processor is configured to perform frequency compounding of the partial-spectrum frames for each of the respective plurality of positions.

9. The system according to claim 4, wherein each of the respective plurality of positions along the slow axis is set at an inter-scan distance L times of an inter-scan distance along the fast axis, wherein 1≤L≤P and P is the number of partial-spectrum frames.

10. The system according to claim 4, comprising at least one processor configured to obtain or derive depth-axis scan information corresponding to the scanning along the fast axis and/or the slow axis, and wherein an inter-scan time between each of the plurality of times is adjustable by varying a period of the depth-axis scan with respect to the slow axis, and/or by varying a number of times the scanner moves along the fast axis and/or the slow axis.

11. (canceled)

12. A method for optical coherence tomography (OCT) for imaging of a sample,
comprising the steps of:
disposing a dispersive element in a sample arm of an optical coherence tomography system to generate an extended source for illuminating the sample;
scanning the extended source across the sample along a fast axis and a slow axis, whereby a plurality of partial-spectrum frames is obtained; and
detecting an interference signal generated by light received from the sample arm and light received from a reference arm of the optical coherence tomography system;
wherein the dispersive element is oriented such that the extended source is disposed at a non-zero angle to the fast axis.

13. The method according to claim 12, wherein the non-zero angle is an acute angle.

14. (canceled)

15. The method according to claim 12, wherein the step of scanning comprises scanning a plurality of times along the fast axis at each of a respective plurality of positions along the slow axis.

16. The method according to claim 12, further comprising a step of generating at least one OCT image and/or at least one optical coherence tomography angiography (OCTA) image from the partial-spectrum frames.

17. The method according to claim 16, further comprising a step of removing low quality frames of the partial spectrum frames prior to generating the at least one OCT image and/or the at least one OCTA image.

18. The method according to claim 16, wherein the step of scanning comprises scanning a plurality of times along the fast axis at each of a respective plurality of positions along the slow axis and wherein generating the OCT image comprises performing temporal averaging of the partial-spectrum frames for each of the respective plurality of positions to generate a plurality of respective OCT images for the respective plurality of positions.

19. The method according to claim 18, further comprises performing frequency compounding of the partial-spectrum frames for each of the respective plurality of positions.

20. The method according to claim 15, wherein each of the respective plurality of positions along the slow axis is set at an inter-scan distance L times of an inter-scan distance along the fast axis, wherein 1≤L≤P and P is the number of partial-spectrum frames.

21. The method according to claim 15, wherein the step of scanning further comprises scanning, obtaining or deriving a depth-axis in combination with the fast axis and/or the slow axis, and wherein an inter-scan time between each of the plurality of times is adjustable by varying a period of scanning the depth-axis with respect to the slow axis and/or by varying a number of times the scanner moves along the fast axis and/or the slow axis.

22. (canceled)

23. A method for modifying an optical coherence tomography (OCT) system for imaging of a sample, the OCT system comprising a sample arm for directing light onto the sample, a reference arm, a detector for detecting an interference signal from light that is reflected from the reference arm and light that is back-reflected or back-scattered from the sample, comprising the steps of:
disposing a dispersive element in the sample arm to generate an extended source for illuminating the sample; and
configuring the scanner to scan the extended source across the sample along a fast axis and a slow axis such that a plurality of partial-spectrum frames is obtained at the detector;
wherein the dispersive element is orientable such that the extended source is disposed at a non-zero angle to the fast axis.
